(19) United States
(12) Patent Application Publication
(10) Pub. No.: US 2010/0172848 A1
(43) Pub. Date: Jul. 8, 2010

(54) SKIN AND SURFACE DISINFECTANT COMPOSITIONS CONTAINING BOTANICALS

(75) Inventors: Shanta Modak, River Edge, NJ (US); Nayana Baiju, New York, NY (US); Lauserpina Caraos, Hollis, NY (US)

Correspondence Address: BAKER BOTTS L.L.P., 30 ROCKEFELLER PLAZA, 44TH FLOOR, NEW YORK, NY, 10112-4498, US

(73) Assignee: The Trustees Of Columbia University In the City of New York, New York, NY (US)

(21) Appl. No.: 12/694,141

(22) Filed: Jan. 26, 2010

Related U.S. Application Data
(63) Continuation of application No. PCT/US08/72006, filed on Aug. 1, 2008.

(51) Int. Cl. A61K 8/97; A61Q 11/00; A61K 36/00; A61K 36/752; A61K 36/54; A01N 65/00; A61Q 17/04; A61Q 5/02; A61Q 19/10; C11D 17/00; A61K 31/075; A01N 65/36

(52) U.S. Cl. 424/58; 424/59; 424/76.1; 424/725; 424/736; 424/739; 424/770; 510/119; 510/130; 510/138; 510/152; 514/717

(57) ABSTRACT

The present invention relates to a skin or surface disinfectant composition with broad spectrum antimicrobial activity comprising one or more essential oil (and/or one or more component thereof) and one or more fruit acid. The compositions of the invention may be used as non-toxic alternatives to conventional disinfectants or may be added to other antimicrobial agents to enhance their activity. The invention provides effective alternatives to harsher products which may be particularly useful in personal care and household products and where children and/or pet exposure may be a concern.

### PRIORITY CLAIMED

This application is a continuation of International Patent Application Serial No. PCT/US08/072006, which claims priority to U.S. patent application Ser. No. 12/134,918, filed Jun. 6, 2008; to U.S. patent application Ser. No. 12/016,788, filed Jan. 18, 2008; which claims priority to U.S. Provisional Application Ser. Nos. 60/953,654, filed Aug. 2, 2007, and 60/945,288, filed Jun. 20, 2007, the disclosures of which are hereby incorporated by reference in their entireties herein.

### GRANT INFORMATION

Not applicable.

### 1. INTRODUCTION

The present invention relates to a skin or surface disinfectant composition with broad spectrum antimicrobial activity comprising one or more essential oil (and/or one or more component thereof) and one or more fruit acid. The compositions of the invention may be used as non-toxic alternatives to conventional disinfectants or may be combined with other antimicrobial agents to enhance their activity. The invention provides effective alternatives to harsher products, and may be particularly useful in personal care and household product applications and where children and/or pet exposure may be a concern.

### 2. BACKGROUND OF THE INVENTION

Essential oils are volatile oils obtained from plant or animal sources and are composed of complex mixtures of several constituents, such as monoterpenes and sesquiterpene hydrocarbons, monoterpene and sesquiterpene alcohols, esters, ethers, aldehydes, ketones, oxides and the like. These essential oils and their isolated constituents are frequently utilized as fragrance and flavor agents, and have been widely used in folk medicine for wound healing properties.

Scientific research has corroborated the beneficial effects of essential oils. Essential oils of eucalyptus have been found to “possess central and peripheral analgesic effects as well as neutrophil-dependent and independent anti-inflammatory activities” (Silva et al., 2003, J. Ethnopharmacol. 89(2-3); 277-283), and similar activity has been observed in essential oils from Lavendula angustifolia Mill. (Hajhashemi et al., 2003, J. Ethnopharmacol. 89(1):67-71). Essential oils have been demonstrated to exhibit antibacterial (Bezic et al., 2003, Phytother. Res. 17(9 :1037-1040; Goren et al., 2003, Z. Naturforsch. 58(9-10):687-690; de Abreu Gonzaga et al., 2003, Planta Med. 69(8:773-775; Valero and Salmera, 2003, Int. J. Food Microbiol. 85(1-2): 73-81) and antifungal (Paranagama et al., 2003, Lett. Appl. Microbiol. 37(1):86-90; Shin, 2003, Arch. Pharm. Res. 26(5):389-393; Velluti et al., 2003, Int. J. Food Microbiol. 89:145-154) activities. Virucidal activity of essential oils has also been observed, including direct virucidal effects against Herpes simplex viruses types 1 and 2 (Garcia et al., Phytother. Res.

17(9):1073-1075; Minami et al., 2003, Microbial Immunol. 47(a):681-684; Schuhmacher et al., 2003, Phytomedicine 10:504-510).

United States Patent Application Publication No. 20050048139 by Modak et al., published Mar. 3, 2005, relates to topical compositions comprising an emollient solvent and an essential oil, which may further comprise additional additives, among which citric acid, glycolic acid and lactic acid are cited. It does not recognize the synergistic activity between essential oils and fruit acids nor does it disclose the concentrations of fruit acids to be used to provide a synergistic effect.

United States Patent Application Publication No. 20050019431 by Modak et al., published Jan. 27, 2005, relates to compositions comprising a quaternary ammonium compound and an essential oil (or active component thereof).

A number of patent applications relate to compositions comprising an essential oil (or component thereof) where zinc salts are added to inhibit irritation associated with essential oils. Examples of such patent applications include United States

Patent Application Publication No. 20040102429 by Modak et al., published May 27, 2004 and United States Patent Application Publication No. 20050238602 by Modak et al., published Oct. 27, 2005.

U.S. Pat. No. 6,858,317 by Aamodt et al., issued Feb. 22, 2005, relates to methods for protecting wood from mold and sapstaining fungi which employ a non-toxic mold inhibitor which may be a plant extract such as an essential oil.

U.S. Pat. No. 5,100,652 by Kross et al., issued Mar. 31, 1992, relates to low concentration chlorous-acid generating oral hygience compositions which may comprise an essential oil as a flavoring agent.

U.S. Pat. No. 5,310,546 by Douglas, issued May 10, 1994, relates to a mouthrinse preparation comprising hydrogen peroxide, zinc chloride, sodium citrate, sodium lauryl sulfate, citric acid and ethanol and optionally an essential oil which is a denaturing agent.

BiON offers several skin care products comprising citric acid, botanicals, and other agents for topical use (San Diego, Calif., US). Johnson et al. (U.S. Pat. No. 6,319,958 and US20020165130) relates to the use of sesquiterpenoids to promote uptake of exogenous antimicrobial compounds. Similarly, a related article discloses the use of sesquiterpenoids, such as nerolidol, farnesol, bisabolol and apritone, in enhancing bacterial permeability and susceptibility to exogenous antimicrobial compounds, suggesting that sesquiterpenoids have a non-specific and general effect (Brehm-Stecher et al. 2003, Antimicrobial Agents and Chemotherapy, 47(10):3357-3360). In particular, Brehm-Stecher et al. report that nerolidol, farnesol, bisabolol and apritone enhanced the susceptibility of S. aureus to the antibiotics erythromycin, gentamicin, vancomycin, ciproflaxin, clindamycin, and tetracycline.

U.S. Pat. No. 4,867,898 by Spaulding et al., issued Sep. 19, 1989, relates to a liquid hard surface cleaner comprising pine oil and organic, oil-soluble acids at a pH from 0-6.

U.S. Pat. No. 6,753,305 by Raso and Caselli, issued Jun. 22, 2004, relates to a hard surface disinfectant comprising up to 20 percent of cinnamon oil or a component thereof, 0.01-5 percent of an organic acid, and optionally an additional essential oil.

International Patent Application Publication No. W02007077573 by Mukhopadhyay, published Jul. 12, 2007, relates to antimicrobial compositions comprising an antimicrobial agent, such as triclosan, and a functionalized hydrocarbon, where the functionalized hydrocarbon can be an essential oil, and/or a solvent.

There is a continuing desire for an antimicrobial composition that is non-irritating, safe, and effective for repeated use in various professional and non-professional settings.

### 3. SUMMARY OF THE INVENTION

The present invention relates to a skin or surface disinfectant composition with broad spectrum antimicrobial activity comprising one or more essential oil (and/or one or more component (i.e., an “Individual Constituent” or “IC”) thereof) and one or more fruit acid. It is based, at least in part, on the discovery that a combination of an essential oil or component thereof together with a fruit acid can confer superior antimicrobial properties on personal care, veterinary, as well as household products. In preferred, non-limiting embodiments, the compositions of the invention further comprise up to about 20 percent alcohol, which facilitates the solubilization of the essential oil(s)/IC(s) and fruit acid. Certain embodiments are also based, at least in part, on the discovery that further addition of an alkanediol, particularly a bifunctional fatty alcohol, enhances antimicrobial activity still more.

In various non-limiting embodiments, the present invention may be utilized in personal care products such as soaps, scrubs, cosmetics, creams and lotions, wound care products, and veterinary products such as pet shampoos, therapeutic ointments, and pet cleansing wipes. In other non-limiting embodiments, the present invention may be utilized in household products such as general purpose cleaning fluids, spray cleaners, laundry detergents, food washes, etc.

The compositions of the invention may be used as non-toxic alternatives to conventional disinfectants or may be combined with to other antimicrobial agents to enhance their activity. The invention provides effective alternatives to harsher products which may be particularly useful in personal care and household products and where children and/or pet exposure may be a concern.

### DETAILED DESCRIPTION OF THE INVENTION

For clarity of description, and not by way of limitation, the detailed description of the invention is divided into the following subsections:

(4.1) essential oils;

(4.2) fruit acids;

(4.3) alkanediols;

(4.4) combinations of essential oils/ICs and fruit acids;

(4.5) compositions comprising alkanediols;

(4.6) personal care products;

(4.7) veterinary products;

(4.8) household/industrial products; and

(4.9) preservative compositions.

### 4.1 Essential Oils

Essential oils (“EOs”), as defined herein, are volatile oils obtained from plant or animal sources, or their synthetic equivalents, and are composed of complex mixtures of several constituents as monoterpenes and sesquiterpene hydrocarbons, monoterpene and sesquiterpene alcohols, esters, ethers, aldehydes, ketones, oxides and the like. Examples of EOs include, but are not limited to, cinnamon oil, basil oil, bergamot oil, clary sage oil, ylang-ylang oil, neroli oil, sandalwood oil, frankincense oil, ginger oil, peppermint oil, lavender oil, jasmine absolute, geranium oil bourbon, spearmint oil, clove oil, patchouli oil, rosemary oil, rosewood oil, sandalwood oil, tea tree oil, vanilla oil, lemongrass oil, cedarwood oil, balsam oils, tangerine oil, Hinoki oil, Hiba oil, ginko oil, eucalyptus oil, lemon oil, orange oil, sweet orange oil, and calendula oil.

In preferred non-limiting embodiments of the invention, the EU is selected from one or more EO from the group consisting of cinnamon oil (bark or leaf), lemongrass oil, citronella oil, basil oil, and orange oil.

Individual constituents (“ICs”) of essential oils may be isolated from the oil (natural) or entirely or partially synthetic, and include, but are not limited to, curcumin, 1-citronellol, α-amylcinnamaldehyde, lyral, geraniol, farnesol, hydroxycitronellal, isoeugenol, eugenol, camphor, eucalyptol, linalool, citral, thymol, limonene and menthol. Further examples of ICs include sesquiterpenoid compounds, which may be the active compounds in the essential oils. Sesquiterpenoid compounds, containing 15 carbons, are formed biosynthetically from three 5-carbon isoprene units. Sesquiterpenoid compounds include, but are not limited to, farnesol, nerolidol, bisabolol, apritone, chamazulene, santalol, zingiberol, carotol, and caryophyllen.

Mixtures of one or more EO, one or more IC, and one or more EO as well as one or more IC, are encompassed by the present invention. In specific non-limiting embodiments of the invention, an IC is selected from the (non-limiting) group consisting of camphor, curcumin, alpha-pinene, constituents of cinnamon leaf oil such as, cinnamaldehyde, cinnamylacetic ester, cinnamic acid, ethyl cinnamate, methyl chavicol, linalool, beta-caryophyllene, and eugenol; constituents of lemongrass oil such as d-limonene, geranyl acetate, nerol, geraniol, citral, and/or myrcene; constituents of citronella oil such as geraniol, citronellol, citronellal, geranyl acetate, limonene, methyl isoueugenol, and/or elemol; components of basil oil such as camphor, limonene, and/or β-selinene; and constituents of orange oil such as α-pinene, sabinene, myrcene, limonene, linalool, citronellal, neral and/or geranial.

An EO or IC for use in the invention may be obtained from its natural source or may be chemically synthesized.

### 4.2 Fruit Acids

Fruit acids which may be used according to the invention include but are not limited to citric acid, glycolic acid, lactic acid, malic acid, tartaric acid and acetic acid. In preferred non-limiting embodiments of the invention, the fruit acid is citric acid.

A fruit acid for use in the invention may be obtained from its natural source or may be chemically synthesized.

### 4.3 Alkanediols

In non-limiting embodiments, bifunctional alcohols which may be used according to the present invention are alkanediols. Suitable alkanediols include, but are not limited to, dodecanediol, decanediol, nonanediol, octanediol, heptanediol, hexanediol and pentanediol.

In particular non-limiting embodiments, the alkanediols have a carbon backbone of between 9 and 25 carbon atoms, including but not limited to 1,9 Nonanediol, 1,2-Decanediol, 1,10-Decanediol, 1,11-Undecanediol, 1,2-Dodecanediol, 1,12 Dodecanediol, Cyclododecanediol, 1,13-Tridecanediol, 1,2-Tetradecanediol, 1,14-Tetradecanediol, 1,15-Pentadecanediol, 1,16-Hexadecanediol, 1,17-Heptadecanediol, 1,18-Octadecanediol, 1,19-Nonadecanediol, 1,20-Eicosanediol, 1,21-Heneicosanediol, 1,22-Docosanediol, 1,23-Tricosanediol, 1,24-Tetracosanediol, 1,25-Pentacosanediol. The preferred alkanediols are 1,2-Decanediol, 1,10-Decanediol, 1,2-Dodecanediol, 1,12-Dodecanediol, Cyclododecanediol, 1,13-Tridecanediol, 1,2-Tetradecanediol, 1,14-Tetradecanediol and the most preferred alkanediols are 1,2-Decanediol, 1,2-Dodecanediol and 1,2-Tetradecanediol.

### 4.4 Combinations of Essential Oils/ICs and Fruit Acids

The present invention provides for compositions comprising a combination of one or more essential oil (and/or one or more IC thereof) and one or more fruit acid. Preferably, this combination produces a synergistic anti-microbial effect against at least one microbe selected from the group consisting of Escherichia coli, Pseudomonas aeruginosa, Staphylococcus aureus, methicillin-resistant S. aureus, and Candida albicans (“synergistic” means that the antimicrobial effect of the combination is greater than the sum of the antimicrobial effects of the individual components).

In particular, non-limiting embodiments of the invention, the compositions comprise between about 0.1 and 1.2 percent (weight/weight) or between 0.1 and 1.0 percent (weight/weight) (“w/w”) of one or more essential oils, one or more ICs, or a combination thereof (where a combination is used, the total of essential oil(s) and/or IC(s) is between about 0.1 and 1.0 percent (weight/weight) and between about 0.125 and 2.0 percent (weight/weight) of one or more fruit acid (where more than one fruit acid is used, the total amount of fruit acids present is between about 0.125 and 2.0 percent (weight/weight)). “About” as used in this document means plus or minus 20 percent of the recited value, so that, for example, “between about 0,125 and 1.0 percent” means a range between 0.125 +.025 and 1.0 +0.2.

In particular, non-limiting embodiments, the present invention provides for concentrates of essential oil/IC/fruit acid combinations which are concentrated and may be diluted to provide a composition for personal, household, or industrial use. In such concentrates, the ratio of fruit acid to essential oil(s)/IC(s) (weight/weight) is between about 1 and 16, for example, but not by way of limitation, fruit acid(s): EO(s)/IC(s) of between about 1:1 to 10:1, inclusive (weight/weight).

The present invention further provides for methods of providing an antimicrobial effect to a surface comprising applying, to the surface, an effective amount of a composition as described herein. An antimicrobial effect means killing and/or inhibiting the growth/proliferation of a microbe. In particular non-limiting embodiments of the invention, the microbe is selected from the group consisting of from the group consisting of Escherichia coli, Pseudomonas aeruginosa, Staphylococcus aureus, methicillin-resistant S. aureus, and Candida albicans. In specific non-limiting embodiments, the composition is exposed to the surface for at least 20 seconds, at least 30 seconds, or at least 60 seconds, or at least 5 minutes or at least 10 minutes. In various non-limiting embodiments, the surface may be the a skin or mucosal surface, a household surface (e.g., a surface of a countertop, Table sink, toilet, wall, floor, appliance, window, shower surface, rug, upholstery, fabric, etc.) or an industrial surface (e.g., a surface of a countertop, Table sink, toilet, wall, floor, appliance, window, shower surface, rug, upholstery, fabric, etc.).

In a first set of specific, non-limiting embodiments, the present invention provides for a composition comprising a component selected from the group consisting of cinnamon oil, cinnamaldehyde, eugenol, cinnamylacetic ester, and cinnamic acid, at a concentration of between about 0.1 and 1.2 percent (weight/weight) or between about 0.2 and 0.6 percent (weight/weight), as well as citric acid at a concentration of between about 0.5 and 1.5 percent (weight/weight), optionally further comprising triclosan at a concentration of between about 0.05 and 3 percent {weight/weight) or between about 0.05 and 0.1 percent (weight/weight) {this range, and all ranges herein, inclusive). In certain embodiments, the EO/IC is not cinnamon oil or pine oil or an IC thereof.

In a second set of non-limiting embodiments, the present invention provides for compositions comprising a EO/IC mixture comprising two or more EO or IC from the group consisting of cinnamon oil or an IC thereof, lemongrass oil and/or an IC thereof, orange oil and/or an IC thereof, basil oil and/or an IC thereof, and citronella oil and/or an IC thereof, at a total EO/IC concentration of between about 0.1 and 1 percent (weight/weight); together with one or more fruit acid (preferably citric acid), at a total fruit acid concentration of between about 0.125 and 2 percent (weight/weight); and an alcohol (preferably ethanol at a concentration of between about 5-20 percent (weight/weight), optionally further comprising triclosan at a concentration of between about 0.05 and 3 percent (weight/weight) or between about 0.05 and 0.1 percent (weight/weight), where the ratio of EO/IC to fruit acid is between about 1:1 to about 1:10. In certain embodiments, the EO/IC is not cinnamon oil or pine oil or an IC thereof.

In a third set of non-limiting embodiments, the present invention provides for compositions comprising a EO/IC mixture comprising lemongrass oil and/or an IC thereof, orange oil and/or an IC thereof, and optionally one or more additional EO and/or IC, at a total EO/IC concentration of between about 0.1 and 1 percent (weight/weight); together with one or more fruit acid (preferably citric acid), at a total fruit acid concentration of between about 0.125 and 2 percent; and an alcohol (preferably ethanol) at a concentration of between about 5-20 percent (weight/weight), optionally further comprising triclosan at a concentration of between about 0.05 and 1 percent (weight/weight) or between about 0.05 and 0.3 percent (weight/weight), where the ratio of EO/IC to fruit acid is between about 1:1 to about 1:10.

### 4.5 Compositions Comprising Alkanediols

In non-limiting embodiments, the present invention provides for compositions comprising an essential oil, a fruit acid, an alcohol which is not an alkanediol, and an alkanediol. In particular, non-limiting embodiments, the carbon backbone of the alkanediol has between 9 and 25 carbon atoms. In particular non-limiting embodiments, the present invention provides for compositions comprising (i) between about 0.2 and 0.7 percent (weight/weight) of one or more essential oil as set forth above and preferably selected from the group consisting of lemongrass, cinnamon oil, citronella oil, basil oil, orange oil and combinations thereof; (ii) a non-alkanediol alcohol solvent at a concentration between about 0.5 and 20 percent (weight/weight); (iii) an amount of alkanediol which increases the antimicrobial effect, for example at a concentration between about 0.3 and 1.0 percent (weight/weight), and (iv) one or more fruit acid at a total concentration between about 0.125 and 2.0 percent (weight/weight).

The preferred essential oils are the ones that show significant enhancement of antimicrobial activity in combination with citric acid. These oils include one or more selected from lemongrass oil, cinnamon oil, basil oil and citronella oil (preferably at a total concentration of between about 0.2 and 0.7 percent (weight/weight), with the optional further addition of orange oil to reduce the pungent odor of the other essential oils and to provide a fragrance which is mild and pleasant. Fruit acids which may be used in such compositions include citric acid or lactic acid (preferably citric acid) at a concentration between about 0.5 and 1.0 percent (weight/weight).

Preferred but non-limiting examples of non-alkanediol alcohols for solubilisation of both essential oils and citric acid are aliphatic alcohols having carbon atoms about 1 to 8 such as methanol, ethanol, n-propanol, isopropyl alcohol, 2-methyl-2 propanol, hexanol, or combinations thereof, at a concentration of between about 5 and 20 percent (weight/weight). Aromatic alcohols, for example, but not by way of limitation, phenoxyethanol, benzyl alcohol, 1-phenoxy-2propanol, and/or phenethyl alcohol, for example at a concentration of between about 0.5 and 5 percent (weight/weight) may also optionally be used in combination with aliphatic alcohols. A further solvent which optionally may be comprised in a composition of the invention is iso propyl myristate. Most preferred aliphatic alcohols include ethanol, denatured alcohol (SDA 40B and SDA3C) and isopropanol. Most preferred aromatic alcohols include phenoxyethanol and phenethanol.

Compositions comprising lemongrass or cinnamon oils (0.2-0.5% (weight/weight)) and orange oil (0.1-0.2% (weight/weight), exhibit a pleasant and mild fragrance. Furthermore these oils even at these lower concentrations have been observed to provide superior antibacterial activity (more than 3 log reduction when challenged with 10⁸ colony forming unit of a gram positive pathogen (S. aureus) in combination with a secondary alcohol (0.3-1.0% (weight/weight)) and alcohol (5-20% (weight/weight)).

In specific, non-limiting embodiments, the present invention provides for a skin or surface disinfectant composition comprising the essential oil lemongrass (0.3-0.5% (weight/weight)), orange oil (0.1-0.2% (weight/weight)), citric acid (0.5-2.0% (weight/weight)), SDA 40B alcohol (5-20% (weight/weight)) and 1,2 decanediol (0.3-1.0% (weight/weight)).

Preferably the pH of personal care products is between about 3.5-5.0, and preferably between about 4-4.7.

In addition to the above ingredients, a composition of the invention may optionally further comprise an emollient to further reduce irritation, such as, but not limited to, a fatty alcohol, behentrimonium methosulfate -cetyl alcohol (Incroquat TMS), or a polyol such as glycerol, propylene glycol, diglycerol, ethylene glycol, diethylene glycol, Methylene glycol, dipropylene glycol, tripropylene glycol, hexylene glycol, butylene glycol, etc.

Essential oils are volatile and therefore it is desirable that the antimicrobial composition containing essential oils is incorporated in a suitable base in which it is stable at higher temperature and over a long period of time. Accordingly, a composition of the invention may optionally comprise a hydrophilic or hydrophobic gel forming polymer, a fatty acids, a plant oils etc. Suitable hydrophilic gel polymers include, but are not limited to, hydroxypropylmethyl cellulose, cationic hydroxyethyl cellulose (U-care polymers), ethyl cellulose, hydroxypropyl cellulose, hydroxymethyl cellulose, carboxy methyl cellulose, polyethylene oxide (polyox resins), and chitosan pyrrolidone carboxylate (Kytamer PC), silica gel, carbomerpolymers etc. Suitable hydrophobic gel polymers include, but are not limited to, silicone polymers, for example polydimethylsiloxane polymer (Dow Corning 225 Silicone Fluid), dimethiconol fluid in dimethicone (Dow Corning 1403 Silicone Fluid), cyclomethicone and dimethicone copolyl (Dow Corning 3225C and Q2-5220 Silicone Fluid), silicone glycol (BASF 1066 DCG polyol), KSG series Silicone gels (Shin-etsu), and combinations thereof Suitable plant oils include, but are not limited to, olive oil, almond oil, avocado oil, basil oil, primrose oil, peanut oil, safflower oil, sesame oil, soyabean oil, wheat germ oil.

### 4.6 Personal Care Products

In non-limiting embodiments, the present invention provides for personal care product compositions comprising a combination of one or more essential oil and/or IC together with one or more fruit acid, as set forth in section 4.4 or 4.5, above. In preferred, non-limiting embodiments, the amounts of the active agents are such that regular exposure of skin to the personal care product does not produce skin irritation in a normal subject.

Non-limiting examples of personal care products which may utilize the invention include bar soap, liquid soap (e.g. hand soap), hand sanitizer, cleansing wipes, body wash, acne treatment products, shampoo, conditioner, cosmetics (including but not limited to liquid or powder foundation, liquid or solid eyeliner, mascara, cream eye shadow, tinted powder, “pancake” type powder to be used dry or moistened, etc.) deodorant, body lotion, hand cream, topical cream, aftershave lotion, skin toner, mouth wash, toothpaste, sunscreen lotion, and baby products such as, but not limited to, cleansing wipes, baby shampoo, baby soap, and diaper cream. The present invention may also be applied to wound care items, such as, but not limited to, wound healing ointments, wound coverings, bandages, tape, and steri-strips, and medical articles such as medical gowns, caps, face masks, and shoe-covers, surgical drops, etc.

Personal care compositions according to the invention, in addition to one or more essential oil and/or IC together with one or more fruit acid, may further comprise one or (preferably) more than one component selected from the group consisting of emollients, stabilizing agents, thickening agents, humectants, anti-inflammatory agents, antimicrobial agents, neutralizing agents, surfactants, water, silicone polymers, alcohols, and hydrogels, as well as additional components as may be known in the art. Non-limiting examples of such components are set forth below.

In various non-limiting embodiments of the invention, a personal care product comprising a combination of one or more essential oil and/or IC together with one or more fruit acid may further comprise an emollient, for example PEG 20 almond glycerides, Probutyl DB-10, Glucam P-20, Glucam E-10, Glucam P-10, Glucam E-20, Glucam P-20 distearate, glycerin, propylene glycol, octoxyglycerine, cetyl acetate, acetylated lanolin alcohol (e.g., Acetulan), cetyl ether (e.g., PPG-10), myristyril ether (e.g., PPG-3), hydroxylated milk glycerides (e.g., Cremeral HMG), polyquaternium compounds (e.g., U-care compounds), copolymers of dimethyl dialyl ammonium chloride and acrylic acid (e.g., Merquat), dipropylene glycol methyl ethers (e.g., Dowanol DPM, Dow Corning), polypropylene glycol ethers (e.g., Ucon 50-HB-600, Union Carbide) and silicon polymers. Other suitable emollients may include hydrocarbon-based emollients such as petrolatum or mineral oil, fatty ester-based emollients, such as methyl, isopropyl and butyl esters of fatty acids such as isopropyl palmitate, isopropyl myristate, isopropyl isostearate, isostearyl isostearate, diisopropyl sebacate, and propylene dipelargonate, 2-ethylhexyl isononoate, 2-ethylhexyl stearate, C₁₂-C₁₆ fatty alcohol lactates such as cetyl lactate and lauryl lactate, isopropyl lanolate, 2-ethylhexyl salicylate, cetyl myristate, oleyl myristate, oleyl stearate, oleyl oleate, hexyl laurate, and isohexyl laurate. Additional useful emollients include lanolin, olive oil, cocoa butter, and shea butter.

In various non-limiting embodiments of the invention, a personal care product comprising a combination of one or more essential oil and/or IC together with one or more fruit acid may further comprise a stabilizing agent consisting of antioxidants, including but not limited to vitamin C (ascorbic acid) and vitamin E (tocopherol), and surfactants, including but not limited to incromide or silicone-based surfactants (Masil SF-19, BASF).

In various non-limiting embodiments of the invention, a personal care product comprising a combination of one or more essential oil and/or IC together with one or more fruit acid may further comprise a thickening and/or gelling agent such as stearyl alcohol, cationic hydroxy ethyl cellulose (Ucare; JR30), hydroxy propyl methyl cellulose, hydroxy propyl cellulose (Klucel), chitosan pyrrolidone carboxylate (Kytamer), behenyl alcohol, zinc stearate, emulsifying waxes, including but not limited to Incroquat and Polawax, an addition polymer of acrylic acid, a resin such as Carbopol® ETD™ 2020, guar gum, acacia, acrylates/steareth-20 methacrylate copolymer, agar, algin, alginic acid, ammonium acrylate co-polymers, ammonium alginate, ammonium chloride, ammonium sulfate, amylopectin, attapulgite, bentonite, C9-15 alcohols, calcium acetate, calcium alginate, calcium carrageenan, calcium chloride, caprylic alcohol, carbomer 910, carbomer 934, carbomer 934P, carbomer 940, carbomer 941, carboxymethyl hydroxyethyl cellulose, carboxymethyl hydroxypropyl guar, carrageenan, cellulose, cellulose gum, cetearyl alcohol, cetyl alcohol, corn starch, damar, dextrin, dibenzlidine sorbitol, ethylene dihydrogenated tallowamide, ethylene diolamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, isocetyl alcohol, isostearyl alcohol, karaya gum, kelp, lauryl alcohol, locust bean gum, magnesium aluminium silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M, PEG-5M, polyacrylic acid, polyvinyl alcohol, potassium alginate, potassium aluminium polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacylate, sodium silicoaluminate, sodium sulfate, stearalkonium bentotnite, stearalkonium hectorite, stearyl alcohol, tallow alcohol, TEA-hydrochloride, tragacanth gum, tridecyl alcohol, tromethamine magnesium aluminium silicate, wheat flour, wheat starch, xanthan gum, abietyl alcohol, acrylinoleic acid, aluminum behenate, aluminum caprylate, aluminum dilinoleate, aluminum salts, such as distearate, and aluminum isostearates, beeswax, behenamide, butadiene/acrylonitrile copolymer, C29-70 acid, calcium behenate, calcium stearate, candelilla wax, carnauba, ceresin, cholesterol, cholesterol hydroxystearate, coconut alcohol, copal, diglyceryl stearate malate, dihydroabietyl alcohol, dimethyl lauramine oleate, dodecanoic acid/cetearyl alcohol/glycol copolymer, erucamide, ethylcellulose, glyceryl triacetyl hydroxystearate, glyceryl tri-acetyl ricinolate, glycol dibehenate, glycol di-octanoate, glycol distearate, hexanediol distearate, hydrogenated C6-14 olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable oil, Japan wax, jojoba wax, lanolin alcohol, shea butter, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhyrdine copolymer, octyldodecyl stearoyl stearate, oleamide, oleostearine, ouricury wax, oxidized polyethylene, ozokerite, paraffin, pentaerythrityl hydrogenated rosinate, pentaerythrityl tetraoctanoate, pentaerythrityl rosinate, pentaerythrityl tetraabietate, pentaerythrityl tetrabehenate, pentaerythrityl tetraoleate, pentaerythrityl tetrastearate, ophthalmic anhydride/glycerinlglycidyl decanoate copolymer, ophthalmic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eiconsene copolymer, PVP/hexadecene copolymer, rice bran wax, stearlkonium bentonite, stearalkonium hectorite, stearamide, stearamide DEA-distearate, stearamide DIBA-stearate, stearamide MEA-stearate, stearone, stearyl erucamide, stearyl stearate, stearyl stearoyl stearate, synthetic beeswax, synthetic wax, trihydroxystearin, triisononanoin, triisostearin, tri-isostearyl trilinoleate, trilaurin, trilinoleic acid, trilinolein, trimyristin, triolein, tripalmitin, tristearin, zinc laurate, zinc myristate, zinc neodecanoate, zinc rosinate, and mixtures thereof. The gelling agents used in vehicles may be natural gelling agents such as natural gums, starches, pectins, agar and gelatin. Often, the gelling agents are based on polysaccharides or proteins Examples include but are not limited to guar gum, Xanthum gum, Alginic acid (E400), sodium alginate (E401), potassium alginate (E402), ammonium alginate (E403), calcium alginate (E404,—polysaccharides from brown algae), Agar (E406, a polysaccharide obtained from red seaweeds), Carrageenan (E407, a polysaccharide obtained from red seaweeds), Locust bean gum (E410, a natural gum from the seeds of the Carob tree), Pectin (E440, a polysaccharide obtained from apple or citrus-fruit), and Gelatin (E441, made by partial hydrolysis of animal collagen).

In various non-limiting embodiments of the invention, a personal care product comprising a combination of one or more essential oil and/or IC together with one or more fruit acid may further comprise a humectant, such as, for example, glycerin, 1-2-propylene glycol, dipropylene glycol, polyethylene glycol, 1,3-butylene glycol, or 1,2,6-hexanetriol.

In certain non-limiting embodiments of the invention, essentially the entire antimicrobial effect of the inventive composition is achieved by an antimicrobial composition consisting of one or more essential oil and/or one or more IC, together with a fruit acid and optionally an alcohol. In alternative embodiments of the invention, one or more additional antimicrobial agent may be comprised, for example, in the amount of between about 0.05 and 2.0 percent (weight/weight), where such antimicrobial agent may be selected from the group consisting of iodophors, iodine, benzoic acid, dihydroacetic acid, propionic acid, sorbic acid, methyl paraben, ethyl paraben, propyl paraben, butyl paraben, cetrimide, benzalkonium chloride, dequalinium chloride, chlorhexidine, chloroeresol, chlorxylenol, benzyl alcohol, bronopol, chlorbutanol, phenoxyethanol, phenylethyl alcohol, 2,4-dichlorobenzyl alcohol, thiomersal, clindamycin, erythromycin, benzoyl peroxide, mupirocin, bacitracin, polymyxin B, neomycin, triclosan, parachlorometaxylene, foscarnet, miconazole, fluconazole, itriconazole, ketoconazole, silver sulfadiazine, octoxyglycerine, biguanides such as, but not limited to, chlorhexidine free base, chlorhexidine palmitate, chlorhexidine diphosphanilate, chlorhexidine digluconate, chlorhexidine diacetate, chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di-α-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine di-2-hydroxynapthoate, chlorhexidine ernbonate, and parahexamethylenebiguanide (“PHMB”).

In various non-limiting embodiments of the invention, a personal care product comprising a combination of one or more essential oil and/or IC together with one or more fruit acid may further comprise a neutralizing agent to neutralize carboxyl groups present in one or more other component, such as carboxyl groups in a thickening agent. Suitable neutralizing agents include diisopropylamine and triethanolamine.

In various non-limiting embodiments of the invention, a personal care product comprising a combination of one or more essential oil and/or IC together with one or more fruit acid may further comprise a surfactant. The surfactant may be an anionic surfactant, a cationic surfactant, an ampholytic surfactant, or a nonionic surfactant. Examples of nonionic surfactants include polyethoxylates, fatty alcohols (e.g., ceteth-20 (a cetyl ether of polyethylene oxide having an average of about 20 ethylene oxide units) and other “BRIJ”® nonionic surfactants available from ICI Americas, Inc. (Wilmington, DE)), cocamidopropyl betaine, alkyl phenols, fatty acid esters of sorbitol, sorbitan, or polyoxyethylene sorbitan. Suitable anionic surfactants include ammonium lauryl sulfate and lauryl ether sulfosuccinate. A preferred surfactant is lauroyl ethylenediamine triacetic acid sodium salt at a concentration between about 0.5-2.0% (weight/weight). In particular non-limiting embodiments of the invention, concentrations of surfactant are between about 0.05% and 2% (weight/weight). In various non-limiting embodiments of the invention, a personal care product may comprise water.

In various non-limiting embodiments of the invention, a personal care product comprising a combination of one or more essential oil and/or IC together with one or more fruit acid may further comprise a hydrogel comprising, for example, a compound such as hydroxypropylmethyl cellulose, cationic hydroxyethyl cellulose (U-care polymers), ethyl cellulose, hydroxypropyl cellulose, hydroxymethyl cellulose, carboxy methyl cellulose, polyethylene oxide (polyox resins), and chitosan pyrrolidone carboxylate (Kytomer PC). In various non-limiting embodiments of the invention, a personal care product comprising a combination of one or more essential oil and/or IC together with one or more fruit acid may further comprise an alcohol or a mixture of alcohols, for example, ethanol, isopropyl alcohol, n-propyl alcohol, and mixtures thereof; fatty alcohols, including, but not limited to, cetyl alcohol, myristol alcohol, stearyl alcohol, octyl alcohol, decyl alcohol and lauryl alcohol, and mixtures thereof; and hexanol.

In various non-limiting embodiments of the invention, a personal care product comprising a combination of one or more essential oil and/or IC together with one or more fruit acid may further comprise a silicone polymer, for example one or more than one polydimethylsiloxane polymer (Dow Corning 225 Silicone Fluid), dimethiconol fluid in dimethicone {Dow Corning 1403 Silicone Fluid), cyclomethicone and dimethicone copolyl {Dow Corning 3225C Silicone Fluid), and silicone glycol (BASF 1066 DCG polyol). In particular, non-limiting embodiments, the amount of silicone polymer is between about 0.1 and 1.0 percent (volume/volume).

In various non-limiting embodiments of the invention, a personal care product comprising a combination of one or more essential oil and/or IC together with one or more fruit acid may further comprise an emollient solvent such as a glycidyl ether having an alkyl chain up to and including 18 carbon molecules and ethoxylates and propoxylates thereof, a glyceryl ether having an alkyl chain up to and including 18 carbon molecules and ethoxylates and propoxylates thereof, a mono- or diglyceryl ether having an alkyl chain up to and including 18 carbon molecules and ethoxylates and propoxylates thereof, ethoxylate and propoxylate ethers, ethoxy diglycol esters, ethyl hexyl alcohol propoxylate, and propylene glycol esther ethoxylates and propoxylates, and Arlamol (Altas).

In various non-limiting embodiments of the invention, a personal care product comprising a combination of one or more essential oil and/or IC together with one or more fruit acid may further comprise additives such as dyes, fragrances, pH adjusters, including basic pH adjusters such as ammonia, mono-, di- and tri-alkyl amines, mono-, di- and tri-alkanolamines, alkali metal and alkaline earth metal hydroxides (e.g., ammonia, sodium hydroxide, potassium hydroxide, lithium hydroxide, monoethanolamine, triethylamine, isopropylamine, diethanolamine and triethanolamine); acid pH adjusters such as mineral acids and polycarboxylic acids (e.g., hydrochloric acid, nitric acid, phosphoric acid, sulfuric acid, citric acid, glycolic acid, and lactic acid); vitamins such as vitamin A, vitamin E and vitamin C; polyamino acids and salts, such as ethylenediamine tetraacidic acid (EDTA), preservatives such as Germall plus and DMDM hydantoin, and sunscreens such as aminobenzoic acid, arobenzone, cinoxate, diioxybenzone, homosalate, menthyl anthranilate, octocrylene, octyl methoxycinnamate, octyl salicylate, oxybenzoate, padimate O, phenylbenzimidazole, sulfonic acid, sulisobenzone, titanium dioxide, trolamine salicylate and zinc oxide.

In one set of non-limiting embodiments, the present invention provides for personal care compositions comprising one or more EO/IC, preferably where the EO(s)/IC(s) are selected from the group consisting of lemongrass oil and/or an IC thereof, orange oil and/or an IC thereof, cinnamon leaf oil and/or an IC thereof, basil oil and/or an IC thereof, eugenol, cinnamaldehyde, cirmamylacetic ester, and cinnamic acid, at a total concentration of between about 0.1 and 1% (weight/weight); a fruit acid, preferably citric acid, at a concentration of between about 0.125 and 1% (weight/weight); an alcohol, preferably ethanol, at a concentration of between about 5 and 20% (weight/weight); and optionally triclosan at a concentration of between about 0.05 and 1% (weight/weight), where the ratio of EO(s)/IC(s) to the fruit acid(s) is between about 1:1 to 1:10 and the pH is between about 3 and about 7, preferably between about 5 and 6.

In another set of non-limiting embodiments, the present invention provides for personal care compositions comprising lemongrass oil or an IC thereof and orange oil or an IC thereof at a total concentration of between about 0.2 and 0.7% (weight/weight); a fruit acid, preferably citric acid, at a concentration of between about 0.25 and 1% (weight/weight); an alcohol, preferably ethanol, at a concentration of between about 5 and 20% (weight/weight); and optionally triclosan at a concentration of between about 0.05 and 1% (weight/weight), where the ratio of EO(s)/IC(s) to fruit acid(s) is between about 1:1 to 1:5 and the pH is between about 3 and about 7, preferably between 5 and 6.

In various non-limiting embodiments of the invention, a personal care product comprising a combination of one or more essential oil and/or IC together with one or more fruit acid may further comprise various anti-inflammatory, antimicrobial agents, anti-irritants, and gelling ingredients. Such compositions may be included in, for example, wound healing ointments. The antimicrobial botanicals contemplated for wound treatment include 0.2-0.7% (weight/weight) essential oils such as lemongrass oil (LG) or orange oil (0), and 0.2-1.0% (weight/weight) fruit acids such as citric acid (Cit) and lactic acid (L), and 0.5-1.0% (weight/weight) phenoxyethanol, which is a constituent of sage oil (PXE). Anti-irritant, anti inflammatory botanicals include, but are not limited to 0.3-0.7% (weight/weight) Calendula oil (Co), 0.1-0.5% (weight/weight) turmeric extract (curcumin (Cr)), 0.2-2.0% (weight/weight) salicylic acid (S), 0.2-0.5% (weight/weight) Camphor (Cm) and 2-30% (weight/weight) honey (H). Gelling agents would include, but are not limited to, Guar gum, Xanthum gum Alginic acid, and Pectin in amounts of 0.2-3.0% (weight/weight).

In one specific, non-limiting embodiment, the present invention provides for a liquid soap product called “CN1-A” having one of the following compositions (CN1-A1 OR CN1-A2).

TABLE 1
CN1-A Compositions

| | Ingredient | % (weight/weight) |
|---|---|---|
| CN1-A1: | Deionized water | 59.15 |
| | Polyox N 60K | 0.2 |
| | Pluronic F 87 Prill | 2.0 |
| | Ucare Jr 30 | 0.4 |
| | D,L Panthenol 50 W | 1.0 |
| | Incromide oxide L | 3.0 |
| | Crosultane C-50 | 3.0 |
| | Montalene C 40 | 3.0 |
| | 2-Phenoxy-ethanol | 1.0 |
| | Zinc gluconate | 0.1 |
| | Glycerine | 2.0 |
| | SDA-40B alcohol | 15.5 |
| | Cinnamon leaf oil | 0.5 |
| | Citric acid | 1.0 |
| | Orange oil | 0.2 |
| | Distilled water | 7.95 |
| CN1-A2: | Deionized water | 59.25 |
| | Polyox N 60K | 0.2 |
| | Pluronic F 87 Prill | 2.0 |
| | Ucare Jr 30 | 0.4 |
| | D,L Panthenol 50 W | 1.0 |
| | Incromide oxide L | 3.0 |
| | Crosultane C-50 | 3.0 |
| | Montalene C 40 | 3.0 |
| | 2-Phenoxy-ethanol | 1.0 |
| | Glycerine | 2.0 |
| | SDA-40B alcohol | 15.5 |
| | Cinnamon leaf oil | 0.5 |
| | Citric acid | 1.0 |
| | Orange oil | 0.2 |
| | Distilled water | 7.95 |

In another specific, non-limiting embodiment, the present invention provides for a liquid soap product called “CN1-B” having the following composition.

TABLE 2
CN1-B

| Ingredient | % (weight/weight) |
|---|---|
| Deionized water | 63.2 |
| Methocel 40-101 | 0.1 |
| Pluronic F 87 Prill | 0.1 |
| Ucare Jr 30 | 0.1 |
| D,L Panthenol 50 W | 1.0 |
| Incromide oxide L | 3.0 |
| Crosultane C-50 | 3.0 |
| Montalene C 40 | 1.5 |
| 2-Phenoxy-ethanol | 1.0 |
| Glycerine | 2.0 |
| SDA-40B alcohol | 15.5 |
| Cinnamon leaf oil | 0.5 |
| Citric acid | 1.0 |
| Orange oil | 0.2 |
| Distilled water | 7.8 |

In another specific, non-limiting embodiment, the present invention provides for a liquid soap product called “CN1-C” having the following composition.

TABLE 3
CN1-C

| Ingredient | % (weight/weight) |
|---|---|
| Deionized water | 63.2 |
| Methocel 40-101 | 0.1 |
| Pluronic F 87 Prill | 0.1 |
| Ucare Jr 30 | 0.1 |
| D,L Panthenol 50 W | 1.0 |
| Incromide oxide L | 3.0 |
| Crosultane C-50 | 3.0 |
| Montalene C 40 | 1.5 |
| 2-Phenyl-ethanol | 1.0 |
| Glycerine | 2.0 |
| SDA-40B alcohol | 15.5 |
| Cinnamon leaf oil | 0.5 |
| Citric acid | 1.0 |
| Orange oil | 0.2 |
| Distilled water | 7.8 |

In a subset of non-limiting embodiments, the present invention provides for a soap comprising one or more essential oil, 1% citric acid, and a soap base comprising a surfactant, an emollient, and a thickener, and having a pH between about 3-5. Specific non-limiting examples of such soaps follow.

TABLE 4
Soap Containing Lemongrass oil, and Citric acid (LG-Cit-4)
(4 represents total oil 0.4%)

| Ingredient | % (w/w) |
|---|---|
| Deionized Water | 63.5 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Lemongrass oil | 0.4 |

TABLE 5
Soap Containing Lemongrass oil, and Citric acid (LG-Cit-6)
(6 represents total oil 0.6%)

| Ingredient | % (w/w) |
|---|---|
| Deionized Water | 63.3 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Lemongrass oil | 0.6 |

TABLE 6
Soap Containing Lemongrass oil, Orange oil (O oil) and Citric acid
(LGO-Cit 6)
(6 represents total oil 0.6%)

| Ingredient | % (w/w) |
|---|---|
| Deionized Water | 63.3 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Lemongrass oil | 0.4 |
| Citric acid | 1.0 |
| Orange oil | 0.2 |

TABLE 7
Soap Containing Lemon grass oil, Orange oil and Citric acid (LGO-Cit 7)
(7 represents total oil 0.7%)

| Ingredient | % (w/w) |
|---|---|
| Deionized Water | 63.2 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Lemongrass oil | 0.5 |
| Citric acid | 1.0 |
| Orange oil | 0.2 |

TABLE 8
Soap Containing Cinnamon oil, Orange oil and Citric acid (CO-Cit 6)
(6 represents total oil 0.6%)

| Ingredient | % (w/w) |
|---|---|
| Deionized Water | 63.3 |
| Methocel 40-101 | 0.1 |
| Pluronic F 87 Prill | 0.1 |
| Ucare Jr 30 | 0.1 |
| DL Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Cinnamon oil | 0.4 |
| Citric acid | 1.0 |
| Orange oil | 0.2 |

TABLE 9
Soap Containing Cinnamon oil, Orange oil and Citric acid (CO-Cit 7)
(7 represent total oil 0.7%)

| Ingredient | % (w/w) |
|---|---|
| Deionized Water | 63.2 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Cinnamon oil | 0.5 |
| Citric acid | 1.0 |
| Orange oil | 0.2 |

TABLE 10
Soap Containing Orange oil and Citric acid (O-Cit 2)
(2 represents total oil 0.2%)

| Ingredient | % (w/w) |
|---|---|
| Deionized Water | 63.7 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Orange oil | 0.2 |
| Citric acid | 1.0 |

TABLE 11
Soap Containing Basil oil (“B oil”), Orange oil (“O oil”) and
Citric acid (BO-Cit 6)
(6 represents total oil 0.6%)

| Ingredient | Percentage (w/w) |
|---|---|
| Deionized Water | 63.3 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Basil oil | 0.4 |
| Citric acid | 1.0 |
| Orange oil | 0.2 |

TABLE 12
Soap containing Citronella oil (“CR oil”), Orange oil (“O oil”),
Citric acid (CRO-Cit6)
(6 represents total oil 0.6%)

| Ingredient | Percentage (w/w) |
|---|---|
| Deionized Water | 63.3 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0 |
| SDA 40B | 15.5 |
| Citronella oil | 0.4 |
| Citric acid | 1.0 |
| Orange oil | 0.2 |

In further specific, non-limiting embodiments, the present invention provides for the following combinations of agents in a soap base (percentages weight/weight):
0.15% TC+0.4% lemongrass oil+0.2% orange oil+1% citric acid;
0.4% lemongrass oil+0.2% orange oil +1% citric acid; or
0.15% TC+0.4% cinnamon oil+0.2% orange oil+1% citric acid; or
0.4% cinnamon oil+0.2% orange oil+1% citric acid.

In still further specific, non-limiting embodiments, the present invention provides for the following combinations of agents in a soap base (percentages w/w):
Cinnamon oil 0.5%+Orange Oil 0.2%+Citric acid 1.0%+alcohol (e.g., denatured ethyl alcohol, such as SDA 40 B) 5.5%+TC 0.14% (or TC 0.15%); or
Lemongrass oil 0.5%+Orange Oil 0.2%+Citric acid 1.0%+alcohol (e.g., denatured ethyl alcohol, such as SDA 40B) 5.5%+TC 0.14% (or TC 0.15%); or
Lemongrass oil 0.5%+Citric acid 1.0%+alcohol (e.g. denatured ethyl alcohol such as SDA 40 B)5.5%+TC 0.14% (or TC 0.15%).

In specific non-limiting embodiments, the present invention provides for compositions comprising (0.2-0.3 percent (weight/weight)) of essential oils such as lemongrass or cinnamon and 0.1-0.2 percent (weight/weight) orange oil when used in combination with 1% citric acid and alkanediols such as 1,2 decanediol, 1,2 dodecanediol and 1,12 dodecanediol, as set forth above. In a specific, non-limiting embodiment, the present invention provides for a soap formulation comprising 0.3% (weight/weight) of lemongrass oil or cinnamon oil in combination with 0.1% (weight/weight) orange oil, and 1% (weight/weight) citric acid with and without alkanediols, where the pH preferably is between 4.5-4.6.

TABLE 13
Soap containing Lemon grass oil, Orange oil and Citric acid
(LG-O-Cit 5) (5 represents total oil 0.5%)

| Ingredient | Percentage (w/w) |
|---|---|
| Deionized Water | 64.8 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.2 |
| pH 4.55 | |

TABLE 14
Soap Containing Lemon grass oil, Orange oil and Citric acid
(LG-O-Cit 4) (4 represents total oil 0.4%)

| Ingredient | Percentage (w/w) |
|---|---|
| Deionized Water | 64.9 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.1 |
| pH 4.64 | |

TABLE 15
Soap Containing LG-O-Cit 5 and 0.3% 1,2 Decanediol

| Ingredient | Percentage (w/w) |
|---|---|
| Deionized Water | 64.5 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.2 |
| 1,2 Decanediol | 0.3 |
| pH 4.6 | |

TABLE 16
Soap Containing LG-O-Cit-4 and 0.3% 1,2 Decanediol

| Ingredient | Percentage (w/w) |
|---|---|
| Deionized Water | 64.6 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.1 |
| 1,2 Decanediol | 0.3 |
| pH 4.6 | |

TABLE 17
Soap Containing LG-O-Cit 4, 0.3% 1,2 Decanediol + 0.5% Incroquat
behenyl TMS

| Ingredient | Percentage (w/w) |
|---|---|
| Deionized Water | 64.1 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.1 |
| 1,2 Decanediol | 0.3 |
| Incroquat TMS | 0.5 |
| pH 4.6 | |

TABLE 18
Soap Containing LG-O-Cit 4 and 0.3% 1,2 Dodecanediol

| Ingredient | Percentage (w/w) |
|---|---|
| Deionized Water | 64.6 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.1 |
| 1,2 Decanediol | 0.3 |
| pH 4.6 | |

TABLE 19
Soap Containing LG-O-Cit 4 and 0.3% 1,12 Dodecanediol

| Ingredient | Percentage (w/w) |
|---|---|
| Deionized Water | 64.6 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.1 |
| 1,12 Dodecanediol | 0.3 |
| pH 4.6 | |

TABLE 20
Soap Containing LG-O-Cit 4 and 0.3% 1,2 tetradecanediol

| Ingredient | Percentage (w/w) |
|---|---|
| Deionized Water | 64.6 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 15.0 |
| Lemon grass oil | 0.3 |
| Citric acid | 1.0 |
| Orange oil | 0.1 |
| 1,2 Tetradecanediol | 0.3 |
| pH 4.6 | |

TABLE 21
Soap Containing LG-O-Cit 4A
(Same as LG-O-Cit 4 but contains 17% SDA-40B alcohol instead of 15%)

| Ingredient | Percentage (w/w) |
|---|---|
| Deionized Water | 62.9 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Lemon grass oil | 0.3 |
| Citric acid | 1.0 |
| Orange oil | 0.1 |
| pH 4.64 | |

TABLE 22
Soap Containing LG-O-Cit 4A and 0.5% 1,2 Decanediol

| Ingredient | Percentage (w/w) |
|---|---|
| Deionized Water | 62.6 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Lemon grass oil | 0.3 |
| Citric acid | 1.0 |
| Orange oil | 0.1 |
| 1,2 Decanediol | 0.5 |
| pH 4.6 | |

TABLE 23
Soap Containing LG-O-Cit 4A and 0.5% 1,2 Dodecanediol

| Ingredient | Percentage (w/w) |
|---|---|
| Deionized Water | 62.6 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Lemon grass oil | 0.3 |
| Citric acid | 1.0 |
| Orange oil | 0.1 |
| 1,2 Dodecanediol | 0.5 |
| pH 4.6 | |

TABLE 24
Soap Containing LG-O-Cit 4A and 0.5% 1,12 Dodecanediol

| Ingredient | Percentage (w/w) |
|---|---|
| Deionized Water | 62.6 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Lemon grass oil | 0.3 |
| Citric acid | 1.0 |
| Orange oil | 0.1 |
| 1,12 Dodecanediol | 0.5 |
| pH 4.6 | |

TABLE 25
Soap Containing LG-O-Cit 4A and 0.5% 1,2 Tetradecanediol

| Ingredient | Percentage (w/w) |
|---|---|
| Deionized Water | 62.6 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Lemon grass oil | 0.3 |
| Citric acid | 1.0 |
| Orange oil | 0.1 |
| 1,2 tetradecanediol | 0.5 |

pH 4.6

TABLE 26
Soap Containing Cn-O-Cit 4A

| Ingredient | Percentage (w/w) |
|---|---|
| Deionized Water | 62.9 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Cinnamon oil | 0.3 |
| Citric acid | 1.0 |
| Orange oil | 0.1 |

pH 4.64

TABLE 27
Soap Containing Cn-O-Cit 4A + 0.5% 1,2 decanediol

| Ingredient | Percentage (w/w) |
|---|---|
| Deionized Water | 62.6 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |
| SDA 40B | 17.0 |
| Cinnamon oil | 0.3 |
| Citric acid | 1.0 |
| Orange oil | 0.1 |
| 1,2 Decanediol | 0.5 |

pH 4.64

TABLE 28
Soap Base

| Ingredient | Percentage (w/w) |
|---|---|
| Deionized Water | 81.3 |
| Methocel 40-101 | 0.2 |
| Pluronic F 87 Prill | 1.0 |
| Polyox WSR-N-60K | 0.2 |
| Ucare Jr 30 | 0.3 |
| D,L Panthenol 50 W | 1.0 |
| Incromide Oxide L | 8.0 |
| Crosultaine C-50 | 3.0 |
| Montalene C 40 | 2.0 |
| 2-Phenoxy-Ethanol | 1.0 |
| Glycerin | 2.0 |

pH 4.64 (adjusted with 10N hydrochloric acid)

TABLE 29
Antibacterial topical lotion comprising LG-O-Cit A + 1,2Decanediol
(“LG-O-Cit A-D Lotion”)

| Ingredient | Percentage (w/w) |
|---|---|
| Water | 65.9 |
| U Care-JR 30M | 0.25 |
| PolyoxWSR-205 | 0.1 |
| Incroquat TMS Behenyl | 2.0 |
| Isopropyl myristate | 1.0 |
| Acetulan | 1.0 |
| Vitamin E | 0.2 |
| Zinc stearate | 0.2 |
| Polawax NF | 2.75 |
| Glycerin | 2.0 |
| Allantoin | 0.2 |
| Dimethicone copolyol (Q2- | 2.5 |
| 5220) | |
| Citric acid | 1.0 |
| 1,2 decanediol | 0.5 |
| Tocopheryl acetate | 0.5 |
| Glyceryl stearate (Arlacel165) | 1.0 |
| Butylene glycol | 3.0 |
| SDA-40-B | 15 |
| Lemongrass oil | 0.5 |
| Tea tree oil | 0.5 |
| Orange oil | 0.1 |
| 1,2 Decanediol(Symclairol) | 0.5 |

(pH adjusted to 4.5-5.0)

TABLE 30
Antibacterial topical lotion comprising LG-O-Cit A + 1,2Decanediol +
Triclosan (“LG-O-Cit A-D-T Lotion”)

| Ingredient | Percentage (w/w) |
|---|---|
| Water | 65.6 |
| U Care-JR 30M | 0.25 |
| PolyoxWSR-205 | 0.1 |
| Incroquat TMS Behenyl | 2.0 |
| Isopropyl myristate | 1.0 |
| Acetulan | 1.0 |
| Vitamin E | 0.2 |
| Zinc stearate | 0.2 |
| Polawax NF | 2.75 |
| Glycerin | 2.0 |
| Allantoin | 0.2 |
| Dimethicone copolyol (Q2- | 2.5 |
| 5220) | |
| Citric acid | 1.0 |
| 1,2 decanediol | 0.5 |
| Tocopheryl acetate | 0.5 |
| Glyceryl stearate (Arlacel165) | 1.0 |
| Butylene glycol | 3.0 |
| SDA-40-B | 15 |
| Lemongrass oil | 0.5 |
| Tea tree oil | 0.5 |
| Orange oil | 0.1 |
| 1,2 Decanediol(Symclairol) | 0.5 |
| Triclosan | 0.3 |

(pH adjusted to 4.5-5.0)

TABLE 31
Antibacterial-anti inflammatory topical lotion comprising
LG-O-CitA + 1,2 Decanediol (“LG-O-Cit A-D AB/AIF Lotion”):

| Ingredient | Percentage (w/w) |
|---|---|
| Water | 65.6 |
| U Care-JR 30M | 0.25 |
| PolyoxWSR-205 | 0.1 |
| Incroquat TMS Behenyl | 2.0 |
| Isopropyl myristate | 1.0 |
| Acetulan | 1.0 |
| Vitamin E | 0.2 |
| Zinc stearate | 0.2 |
| Polawax NF | 2.75 |
| Glycerin | 2.0 |
| Allantoin | 0.2 |
| Dimethicone copolyol (Q2-5220) | 2.5 |
| Citric acid | 1.0 |
| 1,2 decanediol | 0.5 |
| Tocopheryl acetate | 0.5 |
| Glyceryl stearate (Arlacel165) | 1.0 |
| Butylene glycol | 3.0 |
| SDA-40-B | 15 |
| Lemongrass oil | 0.5 |
| Tea tree oil | 0.5 |
| Orange oil | 0.1 |
| 1,2 Decanediol(Symclairol) | 0.5 |
| Curcumin | 0.2 |
| Camphor | 0.1 |

(pH adjusted to 4.5-5.0)

Specific non-limiting examples of antimicrobial formulations follow below.

TABLE 32
Antimicrobial Impregnation solution

| Ingredient | Percentage (w/w) |
|---|---|
| Lemongrass oil | 0.3 |
| Orange oil | 0.1 |
| Tea tree oil | 0.5 |
| Calandula oil | 0.5 |
| Citric acid | 1.0 |
| Olive oil | 5.0 |
| Propylene glycol | 10 |
| Decanediol | 0.5 |
| SDA 40 B alcohol | 51.7 |
| U care JR 30 | 0.4 |
| Water | 30 |

TABLE 33
Antimicrobial/anti-inflammatory Impregnation solution

| Ingredient | Percentage (w/w) |
|---|---|
| Lemongrass oil | 0.3 |
| Orange oil | 0.1 |
| Tea tree oil | 0.3 |
| Calandula oil | 0.5 |
| Citric acid | 1.0 |
| Olive oil | 5.0 |
| Propylene glycol | 10 |
| Decanediol | 0.5 |
| SDA 40 B alcohol | 51.0 |
| U care JR 30 | 0.4 |
| Curcumin | 0.3 |
| Water | 29.7 |

In specific, non-limiting embodiments, the present invention provides for the preparation of topical cream formulations containing anti-irritant, anti-inflammatory agents, gelling agents, and botanicals for minor cuts and wounds. Specific cream formulations are as follows.

TABLE 34
Cream 1 (LGO-L-PXE-Co)

| Ingredients | Percentage (w/w) |
|---|---|
| Water | 65.74 |
| Xanthum gum | 0.5 |
| Glycerin | 15.0 |
| Panthenol (75W) | 0.66 |
| Lemon grass oil | 0.4 |
| Orange oil | 0.1 |
| Calendula oil | 0.5 |
| Lactic acid | 0.5 |
| Phenoxy ethanol | 0.7 |
| Safflower oil | 15.0 |

pH adjusted to 4.5 with 10 N NaOH

TABLE 35
Cream 2 (LGO-L-PXE-Co-S)

| Ingredients | Percentage (w/w) |
|---|---|
| Water | 65.54 |
| Xanthum gum | 0.5 |
| Glycerin | 15.0 |
| Panthenol (75W) | 0.66 |
| Lemon grass oil | 0.4 |
| Orange oil | 0.1 |
| Calendula oil | 0.5 |
| Lactic acid | 0.5 |
| Phenoxy ethanol | 0.7 |
| Safflower oil | 15.0 |
| Salicylic acid | 0.2 |

pH adjusted to 4.5 with 10 N NaOH

TABLE 36
Cream 3 (LGO-L-PXE-Co-Cr)

| Ingredients | Percentage (w/w) |
|---|---|
| Water | 59.25 |
| Xanthum gum | 0.5 |
| Glycerin | 15.0 |
| Panthenol (75W) | 0.66 |
| Lemon grass oil | 0.4 |
| Orange oil | 0.1 |
| Calendula oil | 0.5 |
| Lactic acid | 0.5 |
| Phenoxy ethanol | 0.7 |
| Safflower oil | 15.0 |
| Curcumin | 0.2 |

pH adjusted to 4.5 with 10 N NaOH

TABLE 37
Cream 4 (LGO-L-PXE-Co-Cm-H)

| Ingredients | Percentage (w/w) |
|---|---|
| Water | 55.54 |
| Xanthum gum | 0.5 |
| Glycerin | 15.0 |
| Panthenol (75W) | 0.66 |
| Lemon grass oil | 0.4 |
| Orange oil | 0.1 |
| Calendula oil | 0.5 |
| Lactic acid | 0.5 |
| Phenoxy ethanol | 0.7 |
| Safflower oil | 15.0 |
| Camphor | 0.2 |
| Honey | 10.0 |

pH adjusted to 4.5 with 10 N NaOH

TABLE 38
Cream 5 (LGO-L-PXE-Co-S)

| Ingredients | Percentage (w/w) |
|---|---|
| Water | 65.04 |
| Xanthum gum | 0.5 |
| Glycerin | 15.0 |
| Panthenol (75W) | 0.66 |
| Lemon grass oil | 0.4 |
| Orange oil | 0.1 |
| Calendula oil | 0.5 |
| Lactic acid | 0.5 |
| Phenoxy ethanol | 0.7 |
| Safflower oil | 15.0 |
| Salicylic acid | 0.2 |
| 1,2-decanediol | 0.5 |

pH adjusted to 4.5 with 10 N NaOH

### 4.7 Veterinary Products

In a subset of non-limiting embodiments, the present invention provides for veterinary products comprising a combination of one or more essential oil and/or IC together with one or more fruit acid, as set forth in section 4.4 or 4.5, above. The term “veterinary”, as used here, means “pet care”, and includes home use as well as use in a veterinary office or other pet care establishment.

Non-limiting examples of veterinary care products which may utilize the invention include pet shampoo, pet cleansing wipes including body wipes, ear wipes, and eye wipes, ear cleaning liquid, cage cleaner, surface cleaner for housebreaking accidents, topical creams, ointments, teat dip therapeutic for mastitis and liquid to be applied to pet's skin (as in a “body splash”).

Veterinary care compositions according to the invention, in addition to one or more essential oil and/or IC together with one or more fruit acid, may further comprise one or (preferably) more than one component selected from the group consisting of emollients, stabilizing agents, thickening agents, humectants, antimicrobial agents, neutralizing agents, surfactants, water, silicone polymers, alcohols, and hydrogels, anti-inflammatory agents, wound healing agents, salicylic acid, as well as additional components as may be known in the art.

Specific, non-limiting examples of additional components which may be comprised in pet care products include the components listed above for personal care products.

In certain non-limiting embodiments of the invention, the compositions may be prepared for teat dip to treat mastitis. A general formulation for teat dip compositions is as follows.

TABLE 39
General formulation for teat dip

| Ingredient | Percentage (w/w) |
|---|---|
| anti-irritants | 0.1-5.0% |
| a vehicle containing gelling | 0.2-1.0% |
| agent | |
| glycerin | 5-15% |
| water | 50-80% |
| antimicrobial agents consisting | 0.2-2%), |
| of botanicals | |
| aliphatic and aromatic alcohols | 0.5-15% |
| alkanediol | 0.3-1% |
| solvents such as vegetable oils | |

The anti-irritants used for teat dip may include but are not limited to zinc salts with panthenol, or Bisabolol with ginger root extract (symrelief), or symrelief with a zinc salt. The gelling agents in the vehicle may include but are not limited to natural gelling agents such as natural gums, starches, pectins, agar and gelatin. Antimicrobial botanicals may include but are not limited to lemongrass oil, orange oil and fruit acids such as citric and lactic acid, phenoxyethanol (constituent of sage oil). The following Tables summarize various non limiting examples of formulations.

TABLE 40
Veterinary Composition 1

| Ingredients | Percentage (w/w) |
|---|---|
| Water | 72.14 |
| Xanthum gum | 0.4 |
| Glycerin | 15.0 |
| Zinc gluconate | 0.1 |
| Zinc lactate | 0.1 |
| Zinc acetate | 0.1 |
| Panthenol (75W) | 0.66 |
| Lemon grass oil | 0.3 |
| Orange oil | 0.1 |
| Citric acid | 0.5 |
| Phenoxyethanol | 0.7 |
| Safflower oil | 10.0 |

pH adjusted to 5.0 with 10 N NaOH

TABLE 41
Veterinary Composition 2

| Ingredients | Percentage (w/w) |
|---|---|
| Water | 71.04 |
| Xanthum gum | 0.5 |
| Glycerin | 15.0 |
| Bisabolol, ginger root extract | 0.2 |
| (symrelief) | |
| Panthenol (75W) | 0.66 |
| Lemon grass oil | 0.3 |
| Orange oil | 0.1 |
| Citric acid | 1.0 |
| Phenoxyethanol | 0.7 |
| 1,2 decanediol (symclairol) | 0.5 |
| Safflower oil | 10.0 |

pH adjusted to 5.0 with 10 N NaOH

TABLE 42
Veterinary Composition 3

| Ingredients | Percentage (w/w) |
|---|---|
| Water | 71.04 |
| Xanthum gum | 0.5 |
| Glycerin | 15.0 |
| Bisabolol, ginger root extract | 0.2 |
| (symrelief) | |
| Panthenol (75W) | 0.66 |
| Lemon grass oil | 0.3 |
| Orange oil | 0.1 |
| Lactic acid | 1.0 |
| Phenoxyethanol | 0.7 |
| 1,2 decanediol (symclairol) | 0.5 |
| Fatty acid ester (PCL Liquid 100) | 0.5 |
| Safflower oil | 10.0 |

TABLE 43
Veterinary Composition 4 (teat dip)

| Ingredients | Percentage (w/w) |
|---|---|
| Water | 71.7 |
| Xanthum gum | 0.5 |
| DC Silicone Q2 5220 | 0.5 |
| Glycerin | 15.0 |
| Bisabolol, ginger root extract | 0.2 |
| (symrelief) | |
| Panthenol (75W) | 0.66 |
| Lemon grass oil | 0.3 |
| Orange oil | 0.1 |
| Citric acid | 1.0 |
| Phenoxyethanol | 0.7 |
| 1,2 decanediol (symclairol) | 0.5 |
| Fatty acid ester | 0.5 |
| (PCL Liquid 100) | |
| Safflower oil | 10.0 |

pH adjusted to 5.0 with 10 N NaOH

TABLE 44
Veterinary Composition 5 (teat dip)

| Ingredients | Percentage (w/w) |
|---|---|
| Water | 71.7 |
| Xanthum gum | 0.5 |
| DC Silicone Q2 5220 | 0.5 |
| Glycerin | 15.0 |
| Bisabolol, ginger root extract | 0.2 |
| (symrelief) | |
| Lemon grass oil | 0.3 |
| Orange oil | 0.1 |
| Citric acid | 1.0 |
| Phenoxyethanol | 0.7 |
| 1,2 decanediol (symclairol) | 0.5 |
| Fatty acid ester | 0.5 |
| (PCL Liquid 100) | |
| Safflower oil | 10.0 |

pH adjusted to 5.0 with 10 N NaOH

### 4.8 Household/Industrial Products

In a subset of non-limiting embodiments, the present invention provides for household/industrial products comprising a combination of one or more essential oil and/or IC together with one or more fruit acid, as set forth in section 4.4 and 4.5, above.

Non-limiting embodiments of household/industrial products which may utilize the invention include householder cleaners such as concentrated liquid cleaners and spray cleaners, cleaning wipes, dish washing liquid, dish washer detergent, spray-mop liquid, furniture polish, indoor paint, outdoor paint, dusting spray, laundry detergent, fabric softener, rug/fabric cleaner, window and glass cleaner, toilet bowl cleaner, liquid/cream cleanser, etc. In a particular embodiment, the invention may be used in a food wash product, designed to clean fruits and vegetables prior to consumption. “Household products” are products, other than personal care products, that would be used by individual consumers. “Industrial products” refers to products that are used in industry.

Household-industrial compositions according to the invention, in addition to one or more essential oil and/or IC together with one or more fruit acid, may further comprise one or (preferably) more than one component selected from the group consisting of surfactants, builders (e.g., sequestering builders, precipitating builders, ion exchange builders), solvents, thickeners, abrasives, acids, bases (alkalis), antimicrobial agents, soaps, bleaching agents, enzymes, preservatives, and sudsing agents, as well as additional components as may be known in the art.

In various non-limiting embodiments of the invention, a household/industrial product comprising a combination of one or more essential oil and/or IC together with one or more fruit acid may further comprise a surfactant, for example, but not limited to, an anionic surfactant such as an alkyl sulfate, an alkyldiphenyloxide disulfonate salt (e.g., the DOWFAX series by the Dow Chemical Company), an alkylbenzenesulfonate, an alcohol ethoxysulfate; a cationic surfactant; a non-ionic surfactant, such as a secondary alcohol ethoxylate (e.g., the TERGITAOL series by the Dow Chemical Company) or an alkyl polyglucoside (e.g. the TRITON series by the Dow Chemical Company); or an amphoteric surfactant such as an imidazoline or betaine compound.

In various non-limiting embodiments of the invention, a household/industrial product comprising a combination of one or more essential oil and/or IC together with one or more fruit acid may further comprise a builder, for example, but not limited to, a sequestering builder (chelating agent) such as ethylenediaminetetraacetic acid (“EDTA”), sodium citrate, or a complex phosphate; an ion exchange builder such as zeolite, or a precipitating builder such as sodium carbonate or sodium silicate.

In various non-limiting embodiments of the invention, a household/industrial product comprising a combination of one or more essential oil and/or IC together with one or more fruit acid may further comprise a solvent, for example, but not limited to, water, an alcohol such as methanol, ethanol, isopropyl alcohol, or butanol; a hydrocarbon such as an aromatic hydrocarbon, propylene glycol, methylene chloride, acetone, a petroleum distillate, and/or a glycol ether.

In various non-limiting embodiments of the invention, a household/industrial product comprising a combination of one or more essential oil and/or IC together with one or more fruit acid may further comprise a thickener, for example, but not limited to, a polyethylene glycol. a methoxypolyethylene glycol, and/or hydroxyethyl cellulose.

In various non-limiting embodiments of the invention, a household/industrial product comprising a combination of one or more essential oil and/or IC together with one or more fruit acid may further comprise an abrasive, such as, but not limited to, silica, feldspar or calcite.

In various non-limiting embodiments of the invention, a household/industrial product comprising a combination of one or more essential oil and/or IC together with one or more fruit acid may further comprise an acid, such as, but not limited to, acetic acid, hydroacetic acid, phosphoric acid or hydrochloric acid.

In various non-limiting embodiments of the invention, a household/industrial product comprising a combination of one or more essential oil and/or IC together with one or more fruit acid may further comprise a base (alkali) such as, but not limited to, ammonia or sodium bicarbonate.

In various non-limiting embodiments of the invention, a household/industrial product comprising a combination of one or more essential oil and/or IC together with one or more fruit acid may further comprise an antimicrobial agent, for example, but not limited to, compounds as set forth above for personal care compositions, and also pine oil and sodium hypochlorite.

In various non-limiting embodiments of the invention, a household/industrial product comprising a combination of one or more essential oil and/or IC together with one or more fruit acid may further comprise a bleaching agent, for example, but not limited to, sodium hypochlorite, hydrogen peroxide, sodium percarbonate and sodium perborate.

In various non-limiting embodiments of the invention, a household/industrial product comprising a combination of one or more essential oil and/or IC together with one or more fruit acid may further comprise an enzyme, such as, but not limited to, a protease or a lipase.

In various non-limiting embodiments of the invention, a household/industrial product comprising a combination of one or more essential oil and/or IC together with one or more fruit acid may further comprise a preservative, such as, but not limited to, butylated hydroxytoluene, glutaraldehyde, and EDTA.

In various non-limiting embodiments of the invention, a household/industrial product comprising a combination of one or more essential oil and/or IC together with one or more fruit acid may further comprise a sudsing agent, such as, but not limited to, diethanolamine or triethanolamine.

In one set of non-limiting embodiments, the present invention provides for surface cleaner compositions comprising (i) one or more EO/IC, preferably where the EO(s)/IC(s) are selected from the group consisting of lemongrass oil and/or an IC thereof; orange oil and/or an IC thereof; cinnamon leaf oil and/or an IC thereof; basil oil and/or an IC thereof; and/or pine oil and/or an IC thereof; at a total concentration of between about 0.1 and 1 percent (weight/weight); (ii) a fruit acid, preferably citric acid, at a concentration of between about 1 and 2 percent (weight/weight); (iii) an alcohol, preferably ethanol, at a concentration of between about 5 and 20 percent (weight/weight); and (iv) optionally triclosan at a concentration of between about 0.05 and 1 percent (weight/weight), where the ratio of EO(s)/IC(s) to fruit acid is between about 1:1 to 1:10 (inclusive) and the pH is between about 3 and about 7, preferably between 3 and 5. In certain non-limiting embodiments of the invention, cinnamon leaf oil or an IC thereof and/or pine oil or an IC thereof is not present.

In specific, non-limiting embodiments, the present invention provides for the following surface cleaners, having concentrations of active ingredients as indicated, as well as concentrated stock solutions of these formulations which may be diluted to achieve the respective concentrations.

TABLE 45
Surface Cleaners

| Surface Cleaner | Active ingredients |
|---|---|
| Surface Disinfectant-LG cit 2 | 0.2% Lemon grass oil |
| | 2% Citric acid |
| | 7.65% Alcohol |
| | 0.15% surfactants |
| Surface Disinfectant-LG P cit 4 | 0.3% Pine oil |
| | 0.1% Lemon grass oil |
| | 2% Citric Acid |
| | 7.45% alcohol |
| | 0.15% Surfactants |
| Surface Disinfectant-P cit 5 | 0.5% Pine oil |
| | 2% Citric acid |
| | 7.45% alcohol |
| | 0.15% surfactants |
| Surface Disinfectant-PO Cit 7 | 0.5% Pine oil |
| | 0.2% Orange oil |
| | 1% Citric Acid |
| | 5.35% alcohol |
| | 0.15% Surfactants |
| Surface Disinfectant-LGO Cit 7 | 0.5% lemongrass oil |
| | 0.2% Orange oil |
| | 1% Citric Acid |
| | 5.35% alcohol |
| | 0.15% Surfactants |

TABLE 46
Stock solution of hard surface Disinfectant-LG-O-Cit1 +
Dodecanediol:

| Ingredient | Percentage (w/w) |
|---|---|
| Lemon Grass oil | 3.0 |
| Orange oil | 1.0 |
| Citric Acid | 10.0 |
| 1,12 Dodecanediol | 5.0 |
| SDA 40B alcohol | 79.5 |
| Pluronic Surfactant L-61 | 0.5 |
| Pluronic Surfactant F-127 | 0.5 |
| Pluronic Surfactant F-87 | 0.5 |

Before use, this solution is diluted 10 fold with water.

TABLE 47
Stock solution of hard surface Disinfectant-LG-O-Cit 2 +
Dodecanediol

| Ingredient | Percentage (w/w) |
|---|---|
| Lemon Grass oil | 3.0 |
| Orange oil | 1.0 |
| Citric Acid | 20.0 |
| 1,12 Dodecanediol | 5.0 |
| SDA 40B alcohol | 69.5 |
| Pluronic Surfactant L-61 | 0.5 |
| Pluronic Surfactant F-127 | 0.5 |
| Pluronic Surfactant F-87 | 0.5 |

Before use, this solution is diluted 10 fold with water.

The detailed description hereby incorporates, by reference, the specific working examples of the invention set forth below.

The working examples sometimes refer to Softsoap® or Dial® soaps. Softsoap® is a commercially sold liquid soap comprising water, sodium laureth sulfate, cocamidopropyl betaine, decylglucoside, sodium chloride, fragrance, DMDM hydantoin, PEG-120 methyl glucose dioleate, tetrasodium ethylene diamine tetracetic acid, sodium sulfate, polyquaternium-7, citric acid, poloxamer 124, PEG-7 glyceryl, cocoate, benzophenine-4, and colors.

Dial® soap is a commercially sold liquid soap, where Dial® Antibacterial hand soap comprises, as active agent, 0.15 percent triclosan, and the inactive agents are water, sodium laureth sulfate, ammonium lauryl sulfate, decyl glucoside, cocamidopropyl betaine, glycerine, sodium chloride, PEG-18 gylceryl oleate/cocoate, fragrance, cocamide MEA, DMDM hydantoin, tetrasodium ethylene diamine tetracetic acid and colors.

### 4.9 Preservative Compositions

In certain non-limiting embodiments of the invention, the compositions may be formulated as preservative compositions to be used alone or in conjunction with personal care, household or veterinary, products for preservation purposes. Such compositions may contain lemongrass oil, orange oil, lactic or citric acid, phenoxyethanol and/or an alkanediol. Alkanediols include but are not limited to 1,2-decanediol, 1,12-dodecanediol, and/or 1,2-octanediol. The ingredients are combined in an appropriate solvent including but not limited to ethanol, butanol, 3-methoxy-3-methyl-1-butanol, or combinations thereof. The pH of these solutions are adjusted to 5.0, with an appropriate buffer, including for example sodium hydroxide (NaOH). 0.5-5.0% of the preservative compositions can be used in various formulations, preferably 2.0-3.0% of the preservative compositions.

A general formulation for preservative compositions (which may optimally be in the form of stock solutions, which may be diluted prior to use) is as follows.

TABLE 48
General compositions of preservatives

| | Composition | |
|---|---|---|
| | of stock | Composition in |
| Ingredient | solution % (w/w) | products % (w/w) |
| Lemongrass oil | 10-25 | 0.3-0.5 |
| Orange oil | 1.6-3.3 | 0.05-0.1 |
| Lactic acid/Citric acid | 3.3-13.4 | 0.1-0.4 |
| Phenoxyethanol | 16.6-33.3 | 0-1.0 |
| Alkanediols | 0-35 | 0-1.0 |
| 3-methoxy-3- | 0-50 | 0-1.5 |
| methyl-1-butanol | | |
| SDA 40 B alcohol | 0-70 | 0-2.1 |

Specific non-limiting examples of such preservative formulations follow below.

TABLE 49
Preservative composition A

| Ingredient | Percentage (w/w) |
|---|---|
| Lemongrass oil | 10 |
| Farnesol | 10 |
| Orange oil | 5 |
| lactic acid | 7 |
| 1,2 decanediol | 7 |
| SDA 40 B alcohol | 61 |

TABLE 50
Preservative composition B

| Ingredient | Percentage (w/w) |
|---|---|
| Lemongrass oil | 15 |
| Farnesol | 15 |
| Orange oil | 10 |
| lactic acid | 10 |
| SDA 40 B alcohol | 50 |

TABLE 51
Preservative composition C

| Ingredient | Percentage (w/w) |
|---|---|
| Farnesol | 17 |
| Citric acid | 7 |
| 1,2 decanediol | 7 |
| SDA 40 B alcohol | 69 |

TABLE 52
Preservative composition D

| Ingredient | Percentage (w/w) |
|---|---|
| Lemongrass oil | 15 |
| Orange oil | 5 |
| Lactic acid | 10 |
| 1,2 decanediol | 20 |
| 1,2 Octanediol | 20 |
| SDA 40 B alcohol | 30 |

TABLE 53
Preservative composition E

| Ingredient | Percentage (w/w) |
|---|---|
| Lemongrass oil | 15 |
| Orange oil | 5 |
| Lactic acid | 10 |
| 1,2 Octanediol | 40 |
| SDA 40 B alcohol | 30 |

Additional specific non-limiting examples of preservative compositions follow below, with the compositions of stock solutions as well as varying percentages of the preservative compositions in products.

TABLE 54
Preservative composition F

| | Composition of stock | Products containing 2% |
|---|---|---|
| Ingredient | solution % (w/w) | of preservative % (w/w) |
| Lemongrass oil | 25 | 0.5 |
| Orange oil | 5 | 0.1 |
| Lactic acid | 10 | 0.2 |
| Phenoxyethanol | 35 | 0.7 |
| SDA 40 B alcohol | 25 | 0.5 |

TABLE 55
Preservative composition G

| | Composition of stock | Products containing 3% |
|---|---|---|
| Ingredient | solution % (w/w) | of preservative % (w/w) |
| Lemongrass oil | 10 | 0.3 |
| Orange oil | 3.3 | 0.1 |
| Lactic acid | 6.7 | 0.2 |
| Phenoxyethanol | 23.3 | 0.7 |
| 1,2-decanediol | 16.7 | 0.5 |
| SDA 40 B alcohol | 40 | 1.2 |

TABLE 56
Preservative composition H

| | Composition | Products containing |
|---|---|---|
| | of stock | 3% of preservative |
| Ingredient | solution % (w/w) | % (w/w) |
| Lemongrass oil | 10 | 0.3 |
| Orange oil | 3.3 | 0.1 |
| Lactic acid | 6.7 | 0.2 |
| Phenoxyethanol | 23.3 | 0.7 |
| 1,12-dodecanediol | 16.7 | 0.5 |
| 3-methoxy-3-methyl-1-butanol | 40 | 1.2 |

TABLE 57
Preservative composition I

| | Composition | Products containing |
|---|---|---|
| | of stock | 3% of preservative |
| Ingredient | solution % (w/w) | % (w/w) |
| Lemongrass oil | 16.7 | 0.5 |
| Orange oil | 3.3 | 0.1 |
| Lactic acid | 6.7 | 0.2 |
| Phenoxyethanol | 23.3 | 0.7 |
| 1,12-dodecanediol | 16.7 | 0.5 |
| 3-methoxy-3-methyl-1-butanol | 33.3 | 1.0 |

TABLE 58
Preservative composition J

| | | Products containing |
|---|---|---|
| | Composition of stock | 2.5% of preservative |
| Ingredient | solution % (w/w) | % (w/w) |
| Lemongrass oil | 16.7 | 0.5 |
| Orange oil | 3.3 | 0.1 |
| Lactic acid | 6.7 | 0.2 |
| 1,12-dodecanediol | 33.3 | 0.5 |
| 3-methoxy-3-methyl-1- | 40 | 1.2 |
| butanol | | |

TABLE 59
Preservative composition K

| | | Products containing |
|---|---|---|
| | Composition of stock | 2.5% of preservative |
| Ingredient | solution % (w/w) | % (w/w) |
| Lemongrass oil | 20 | 0.5 |
| Orange oil | 4 | 0.1 |
| Lactic acid | 8 | 0.2 |
| Octanediol | 40 | 1.0 |
| SDA 40 B | 28 | 0.7 |

TABLE 60
Preservative composition L

| | Composition of stock | Products containing 3% |
|---|---|---|
| Ingredient | solution % (w/w) | of preservative % (w/w) |
| Lemongrass oil | 16.7 | 0.5 |
| Orange oil | 3.3 | 0.1 |
| Lactic acid | 6.7 | 0.2 |
| 1,12-decanediol | 33.3 | 1.0 |
| SDA 40 B | 40.0 | 1.2 |

TABLE 61
Preservative composition M

| | Composition | Products containing |
|---|---|---|
| | of stock | 3% of preservative |
| Ingredient | solution % (w/w) | % (w/w) |
| Lemongrass oil | 10 | 0.3 |
| Orange oil | 3.3 | 0.1 |
| Farnesol | 10 | 0.3 |
| Lactic acid | 6.7 | 0.2 |
| Phenoxyethanol | 20 | 0.6 |
| 1,12-dodecanediol | 16.7 | 0.5 |
| 3-methoxy-3-methyl-1-butanol | 33.3 | 1.0 |

TABLE 62
Preservative composition N

| | Composition of stock | Products containing 2% |
|---|---|---|
| Ingredient | solution % (w/w) | of preservative % (w/w) |
| Lemongrass oil | 25 | 0.5 |
| Orange oil | 5 | 0.1 |
| Citric acid | 25 | 0.5 |
| SDA 40 B | 45 | 0.9 |

TABLE 63
Preservative composition O

| | | Products containing |
|---|---|---|
| | Composition of stock | 2.5% of preservative |
| Ingredient | solution % (w/w) | % (w/w) |
| Lemongrass oil | 20 | 0.5 |
| Orange oil | 4.0 | 0.1 |
| Citric acid | 20 | 0.5 |
| Phenoxyethanol | 28 | 0.7 |
| SDA 40 B | 28 | 0.7 |

TABLE 64
Preservative composition P

| | | Products containing |
|---|---|---|
| | Composition of stock | 2.5% of preservative |
| Ingredient | solution % (w/w) | % (w/w) |
| Lemongrass oil | 20 | 0.5 |
| Orange oil | 4.0 | 0.1 |
| Citric acid | 20 | 0.5 |
| 1,2-octanediol | 28 | 0.7 |
| SDA 40 B | 28 | 0.7 |

TABLE 65
Preservative composition Q

| | | Products containing |
|---|---|---|
| | Composition of stock | 2.5% of preservative |
| Ingredient | solution % (w/w) | % (w/w) |
| Lemongrass oil | 20 | 0.5 |
| Orange oil | 4.0 | 0.1 |
| Citric acid | 20 | 0.5 |
| 1,2-decanediol | 28 | 0.7 |
| SDA 40 B | 28 | 0.7 |

### 5. EXAMPLE 1

Various concentrations of basil oil and acetic, lactic, and citric acids, separately and in combination, were prepared in 10 percent SDA40-B alcohol and water, and adjusted to 100 percent. Except for citric acid, which was added by weight, all other ingredients were measured by volume. 0.9 ml of each solution were dispensed in sterile culture tubes, in triplicate, and 0.1 ml of a 10⁷ cfu/ml S. aureus culture was added to the tubes, vortexed, and then, five minutes later, 9.0 ml of drug inactivating medium was added to each tube. Serial dilutions were made with the drug inactivating medium. 0.5 ml of the dilutions were plated on trypticase soy agar (“TSA”) plates. As a control, water containing 10 percent SDA40-B alcohol was processed in parallel. The plates were incubated at 37° C. for 24-48 hours and then the colony counts were determined. The results are shown in Table 66. The greater synergy was observed between basil oil and citric acid (“CA”).

TABLE 66

| Compounds | Log 10 Reduction* |
|---|---|
| 1% Basil oil | 2.7 |
| 0.5% Basil oil | 1.8 |
| 0.25% Basil oil | 0.4 |
| 1% Acetic acid (AA) | 0.2 |
| 1% Lactic acid (LA) | 2.3 |
| 1% Citric Acid (CA) | 0.1 |
| 1% Basil oil + 1% LA | 5.4 |
| 1% Basil oil + 1% AA | 3.4 |
| 1% Basil oil + 1% CA | 5.1 |
| 0.5% Basil oil + 1% CA | 5.1 |
| 0.5% Basil oil + 0.5% CA | 5.0 |
| 0.25% Basil oil + 1% CA | 5.0 |
| 0.25% Basil oil + 0.5% CA | 2.5 |

*Log reduction from control bacterial counts ranging from 1 × 10⁶ to 5 × 10⁶.

The same methodology was used to test the antimicrobial activity of combinations of citric acid with other essential oils. The results are shown in Table 67. In these experiments, cinnamon oil and citronella oil exhibited superior antimicrobial activities in combination with citric acid.

TABLE 67

| Compounds | Log 10 Reduction* |
|---|---|
| 1% CA | 0.1 |
| 0.5% Cinnamon bark | 2.4 |
| 0.25% Cinnamon bark | 1.1 |
| 0.5% Cinnamon bark + 1% CA | 5.9 |
| 0.25% Cinnamon bark + 1% | 4.3 |
| CA | |
| 0.125% Cinnamon bark + 1% | 2.6 |
| CA | |
| 0.25% Cinnamon leaf | 2.8 |
| 0.25% Cinnamon leaf + 1% | 5.7 |
| CA | |
| 0.125% Citronella oil | 1.3 |
| 0.25% Citronella oil + 1% CA | 6.2 |
| 0.125% Citronella oil + 1% | 3.3 |
| CA | |
| 0.25% Orange oil | 0 |
| 0.25% Orange oil + 1% CA | 2.3 |
| 0.25% Lemon oil | 0.05 |
| 0.25% Lemon oil + 1% CA | 3.3 |
| 0.25% Lavender oil | 0.25 |
| 0.25% Lavender oil + 1% CA | 4.0 |
| 0.25% Clove oil | 0.1 |
| 0.25% Clove oil + 1% CA | 3.3 |
| 0.25% Tea tree oil | 0 |
| 0.25% Tea tree + 1% CA | 4.7 |
| 0.25% Farnesol | 0 |
| 0.25% Farnesol + 1% CA | 4.0 |

* Log reduction from control bacterial counts ranging from 1 × 10⁶ to 5 × 10⁶.

Next, the same general protocol was used to test the efficacy of basil, cinammon and citronella oils against a variety of organisms, namely E. coli, P. aeruginosa, MRSA, C. albicans, and S. aureus. The results, which demonstrates that in these experiments, combinations of cinnamon oil and citric acid exhibited superior antimicrobial action, are shown in Table 68.

TABLE 68
Log 10 Reductions*

| Compounds | E. coli | P. aeruginosa | MRSA | C. albicans | S. aureus |
|---|---|---|---|---|---|
| 0.25% Basil oil + 1% CA | 6.0 | 5.5 | 5.2 | 1.5 | 5.0 |
| 0.25% Cinnamon bark oil + 1% CA | 6.0 | 6.0 | 5.2 | 4.5 | 4.3 |
| 0.25% Citronella oil + 1% CA | 6.4 | 6.4 | 6.5 | 1.0 | 6.3 |
| 0.25% Cinnamon leaf oil + 1% CA | 6.4 | 6.4 | 6.5 | 5.4 | 6.3 |
| 0.25% Eugenol + 1% CA | — | 6.5 | — | 5.5 | — |

* Log reduction from control bacteria counts ranging from 1 × 10⁶ to 5 × 10⁶ or C. albicans ranging from 1 × 10⁵ to 5 × 10⁵.

### 6. EXAMPLE 2

The following experiments were performed to evaluate the effectiveness of a hard surface cleaner composition comprising cinnamon leaf oil and citric acid.

Two stock solutions of a hard surface cleaner/disinfectant was prepared, with the following ingredients (the two solutions contained different amounts of cinnamon leaf oil, and therefore the amount of alcohol to bring the solution to 100% also varied).

TABLE 69

| Ingredient | Percentage (w/w) |
|---|---|
| Cinnamon leaf oil | 3.6 or 7.2% |
| Citric acid | 14.3% |
| SDA 40B alcohol | 77.2 or 75.49% |
| | (to bring the volume to 100%) |
| Pluronic surfactant L-61 | 0.7% |
| Pluronic surfactant F-127 | 0.7% |
| Pluronic surfactant F-87 | 0.7% |
| Orange oil | 2.8% |

7% of the stock hard disinfectant was diluted with water to 100%.

0.1 ml of culture containing approximately 1×10⁷ colony forming units (“cfu”) per milliliter was spread evenly on the surface of 2.5×11 cm² tiles using a glass rod and left at room temperature for 10 minutes to dry. After 10 minutes 0.3 ml of the diluted surface disinfectant was spread evenly on the tiles with a sterile glass rod and left for another 10 minutes to dry. The tiles were rinsed with 9.6 ml of inactivating medium (BPBNS), which was collected for testing. The collected medium was serially diluted and 0.5 ml was plated onto TSA plates and incubated at 37° C. for 18-24 hours. The colonies on the plates were counted and the values converted to log₁₀. Commercially available Pinesol®, which contains pine oil, was used as a basis for comparison. Pinesol® containing 15% pine oil was diluted with water as per the manufacturer's instructions to a final concentration of 0.9% pine oil. The results are shown in Table 70. The results show that the composition comprising 0.5% cinnamon leaf oil and 1% citric acid exhibited greater antimicrobial activity than the pine oil cleaner against 4 out of 5 microbes tested.

TABLE 70
Log 10 Reductions*

| | 0.25% cinn. oil + | 0.5% cinn. oil + | |
|---|---|---|---|
| Organism | 1% CA | 1% CA | 0.1% Pinesol ® |
| E. coli | 5.3 | 5.3 | 5.7 |
| P. aeruginosa | 6.1 | 6.1 | 3.9 |
| MRSA | 2.3 | 3.4 | 2.2 |
| C. albicans | 2.5 | 5.2 | 2.1 |
| S. aureus | 3.7 | 4.1 | 2.4 |

*log₁₀ reduction from control bacterial counts (ranges from 1 × 10⁶-5 × 10⁶ for all bacteria, but for C. albicans counts were 1 × 10⁵-5 × 10⁵.

### 7. EXAMPLE 3

Various concentrations of cinnamon leaf oil and citric acid were dissolved in SDA 40-B alcohol (10%) and water, and adjusted to 100 percent. Except for citric acid, which was added by weight, all other ingredients were measured by volume. 0.9 ml of each solution were dispensed in sterile culture tubes, in triplicate, and 0.1 ml of 10⁷ cfu/ml of S. aureus culture was added to the tubes, vortexed, and then, five minutes later, 9.0 ml of drug inactivating medium was added to each tube. Serial dilutions were made with the drug inactivating medium. 0.5 ml of the dilutions were plated on trypticase soy agar (“TSA”) plates. As a control, water containing 10% percent SDA40-B alcohol was processed in parallel. The plates were incubated at 37° C. for 24-48 hours and then the colony counts were determined. The results are shown in Table 71.

TABLE 71

| Compounds | Log 10 reduction |
|---|---|
| Citric Acid 2% | 0.32 |
| Citric Acid 1.0% | 0.30 |
| Citric Acid 0.5% | 0.20 |
| Citric Acid 0.25% | 0.08 |
| Citric Acid 0.125% | 0.02 |
| Cinnamon leaf oil 0.25% | 0.52 |
| Cinnamon leaf oil 0.5% | 0.55 |
| 0.25% Cinnamon + 0.25% CA | 0.73 |
| 0.25% Cinnamon + 0.5% CA | 3.0 |
| 0.25% Cinnamon + 1.0% CA | 5.6 |
| 0.5% Cinnamon + 0.125% CA | 0.84 |
| 0.5% Cinnamon + 0.25% CA | 2.2 |
| 0.5% Cinnamon + 0.5% CA | 3.2 |
| 0.5% Cinnamon + 1.0% CA | 6.5 |
| 0.5% Cinnamon + 2.0% CA | 6.7 |

### 8. EXAMPLE 4

A liquid soap, called “CN1-A” containing cinnamon oil and citric acid was prepared, having the following composition.

TABLE 72

| Ingredient | Percentage (w/w) |
|---|---|
| Deionized water | 59.15% |
| Polyox N 60K | 0.2% |
| Pluronic F 87 Prill | 2.0% |
| Ucare Jr 30 | 0.4% |
| D,L Panthenol 50 W | 1.0% |
| Incromide oxide L | 3.0% |
| Crosultane C-50 | 3.0% |
| Montalene C 40 | 3.0% |
| 2-Phenoxy-ethanol | 1.0% |
| Glycerine | 2.0% |
| SDA-40B alcohol | 15.5% |
| Cinnamon leaf oil | 0.5% |
| Citric acid | 1.0% |
| Orange oil | 0.2% |
| Distilled water | 7.95% |

To prepare the soap, cinnamon oil orange oil, citric acid, and phenoxyethanol are dissolved in the alcohol, the remaining ingredients are dissolved in/mixed with water, and then the alcohol and water solutions are mixed. The pH of the mixture was then adjusted to between 5.5 and 6.5 with 0.1 N NaOH.

The antimicrobial activity of the above soap was tested in parallel with commercial Softsoap® containing triclosan (Softsoap Antibacterial; Colgate-Palmolive). 0.1 ml of a 10⁸ cfu/ml culture of each microbe tested was mixed with 0.1 ml of bovine serum and placed in a sterile culture tube. 0.8 ml of the test soap formulation was added to the tube and vortexed for 30 seconds. Then 9.0 ml DNB was added to neutralize the activity of the soap. The tube was then vortexed and serially diluted with DNB. 0.5 ml of the diluted solution was plated on TSA plates. The same soap base lacking cinnamon oil, citric acid, and orange oil, with phosphate buffered saline mixed with the culture, were used as the controls. The results are shown in Table 73.

TABLE 73

Log₁₀ reduction
from control*

| | | Softsoap ® |
|---|---|---|
| Organisms | CN-1A | (0.15% TC) |
| S. aureus | 2.0 | 0.33 |
| P. aeruginosa | 2.5 | 0.6 |
| E. coli | 4.86 | 0.5 |
| MRSA | 2.7 | 0.8 |
| C. albicans | 1.43 | 0.0 |

*log₁₀ reduction from control microbe counts which in all cases ranged from 1 × 10⁷-5 × 10⁷.

### 9. EXAMPLE 5

A liquid soap, called “CN1-B” containing cinnamon oil and citric acid was prepared, having the following composition.

TABLE 74

| Ingredient | Percentage (w/w) |
|---|---|
| Deionized water | 63.2% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerine | 2.0% |
| SDA-40B alcohol | 15.5% |
| Cinnamon leaf oil | 0.5% |
| Citric acid | 1.0% |
| Orange oil | 0.2% |
| Distilled water | 7.8% |

To prepare the soap, cinnamon oil orange oil, citric acid, and phenoxyethanol are dissolved in the alcohol, the remaining ingredients are dissolved in/mixed with water, and then the alcohol and water solutions are mixed. The pH of the mixture was then adjusted to between 5.5 and 6.5 with 0.1 N NaOH.

The antimicrobial activity of the above soap was tested in parallel with commercial Dial® Antibacterial Hand Soap) containing triclosan. 0.1 ml of a 10⁸ cfu/ml culture of each microbe tested was mixed with 0.1 ml of bovine serum and placed in a sterile culture tube. 0.8 ml of the test soap formulation was added to the tube and vortexed for 30 seconds. Then 9.0 ml DNB was added to neutralize the activity of the soap. The tube was then vortexed and serially diluted with DNB. 0.5 ml of the diluted solution was plated on TSA plates. The same soap base lacking cinnamon oil, citric acid, and orange oil, with phosphate buffered saline mixed with the culture, were used as the controls. The results are shown in Table 75.

TABLE 75

Log₁₀ reduction
from control*

| | | Dial ® soap |
|---|---|---|
| Organisms | CN1-B | (0.15% TC) |
| S. aureus | 5.0 | 0.36 |
| MRSA | 5.1 | 0.03 |
| E. coli | 4.45 | 0 |
| P aeruginosa | 5.9 | 0.12 |

*log₁₀ reduction from control microbe counts which in all cases ranged from 1 × 10⁷ to 5 × 10⁷ 3.4 × 10⁶ for S. aureus, 3-5 × 10⁶ for E. coli and 6 × 10⁵-1.3 × 10⁶ for MRSA.

### 10. EXAMPLE 6

The effectiveness of Softsoap® Juicy Melon (Colgate-Palmolive) with added cinnamon oil, citric acid, and/or triclosan, against MRSA was evaluated. Testing was performed essentially as set forth in the preceding section 9. The results are shown in Table 76.

TABLE 76

| Compounds | Log 10 reduction* |
|---|---|
| Soft Soap ® + 1.5% cin-cit | 3.63 |
| Soft soap ® + 0.075% TC | 0.15 |
| Soft soap ® + 0.15% TC | 0.20 |
| Soft soap ® + 0.3% TC | 0.58 |
| Soft soap ® + 0.075% TC + 1.5% Cin- | 4.29 |
| Cit | |
| Soft soap ® + 0.15% TC + 1.5% Cin-Cit | 4.87 |
| Soft soap ® + 0.3% TC + 1.5% Cin-Cit | 6.38 |

*log₁₀ reduction from control microbe counts which in all cases ranged from 1 × 10⁶-5 × 10⁶.

### 11. EXAMPLE 7

The ability of cinnamon oil and citric acid to potentiate the activity of commercial triclosan-containing soaps such as Softsoap® and Dial® Antibacterial Hand Soap containing 0.15% triclosan was tested using an assay essentially as set forth in Section 9, above. The results are shown in Table 77.

TABLE 77

| | Log₁₀ reduction from control* | | |
|---|---|---|---|
| | S. aureus | E. Coli | MRSA |
| Soft Soap ®-TC | 0.33 | 0.25 | 0.37 |
| Soft Soap ®-TC + CIN- | 3.9 | 3.93 | 6.0 |
| Cit | | | |
| Dial ® Soap-TC | 0.36 | 0 | 0.24 |
| Dial ® Soap-TC + Cin-Cit | 3.74 | 4.18 | 6.0 |

*Log reduction from control bacterial counts (ranges from 3 4 × 10⁶ for S. aureus, 3-5 × 10⁶ for E. coli and 6 × 10⁵-1.3 × 10⁶ for MRSA.

In these experiments, the combination of cinnamon oil and citric acid was found to substantially improve the antimicrobial activity of the commercial soap.

### 12. EXAMPLE 8

Because a major ingredient of cinnamon oil is eugenol, the effect of adding eugenol on the antimicrobial activity of commercial soaps was also tested. The assay was essentially as set forth in Section 9, above. The results are shown in Table 78.

TABLE 78

| | Log reduction from control* |
|---|---|
| | S. aureus |
| Dial ® Soap-TC | 0.30 |
| Dial ® Soap-TC + 0.5% | 2.32 |
| Eugenol + 1% CA | |
| Dial ® Soap-TC + 0.0.5% | 3.94 |
| cinnamon oil + 1% CA | |

*Log reduction from control bacterial counts (ranged from 3-4 × 10⁶ for S. aureus).

These experiments showed that while adding eugenol improved the antimicrobial effect, the improvement was not as great as that observed for cinnamon oil.

### 13. EXAMPLE 9

The following experiments were performed to evaluate the antibacterial activity of LG and Citric acid dissolved in alcohol, where the test organism used was S. aureus. Various amounts of LG oil and Citric acid were dissolved in SDA40-B alcohol, and then water was added to result in the EO concentration shown and an alcohol concentration of 10 percent. 0.9 ml of each solution were dispensed in sterile culture tubes, in triplicate, and 0.1 ml of a 10⁷ cfulml S. aureus culture was added to the tubes, vortexed, and then, five minutes later, 9.0 ml of drug inactivating medium was added to each tube. Serial dilutions were made with drug inactivating medium. 0.5 ml of the dilutions were plated on trypticase soy agar (“TSA”) plates. As a control, water containing 10 percent SDA40-B alcohol was processed in parallel. The plates were incubated at 37° C. for 24-48 hours and then the colony counts were determined. The results are shown in Table 79.

TABLE 79

| | Log 10 reduction |
|---|---|
| Compounds | from control |
| 1% Citric acid | 0.3 |
| 0.5% LG oil | 1.24 |
| 0.55 LG oil + 1% Citric acid | 5.59 |

*Log 10 reduction from control bacterial counts (control counts ranges from 1 × 10⁶ to 5 × 10⁶)

The results shown in Table 79 indicate that LG oil exhibits superior anti bacterial action in combination with Citric acid.

### 14. EXAMPLE 10

Soaps were prepared containing one or more essential oil, 1% citric acid, and a soap base containing surfactants, emollients, thickeners etc. The pH of the Soaps ranged from 3.2-3.3.

TABLE 80
Soap Containing Lemongrass oil, and Citric acid (LG-Cit-4)
(4 represents total oil 0.4%)

| Ingredient | Percentage (w/w) |
|---|---|
| Deionized water | 63.5% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Lemongrass oil | 0.4% |

TABLE 81
Soap Containing Lemongrass oil, and Citric acid (LG-Cit-6)
(6 represents total oil 0.6%)

| Ingredient | Percentage (w/w) |
|---|---|
| Deionized water | 63.3% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Lemongrass oil | 0.6% |

TABLE 82
Soap Containing Lemongrass oil, Orange oil (O oil) and
Citric acid (LGO-Cit 6) (6 represents total oil 0.6%)

| Ingredient | Percentage (w/w) |
|---|---|
| Deionized water | 63.3% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Lemongrass oil | 0.4% |
| Citric acid | 1.0% |
| Orange oil | 0.2% |

TABLE 83
Soap Containing Lemon grass oil, Orange oil and Citric acid
(LGO-Cit 7) (7 represents total oil 0.7%)

| Ingredient | Percentage (w/w) |
|---|---|
| Deionized water | 63.2% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Lemongrass oil | 0.5% |
| Citric acid | 1.0% |
| Orange oil | 0.2% |

TABLE 84
Soap Containing Cinnamon oil, Orange oil and Citric acid
(CO-Cit 6) (6 represents total oil 0.6%)

| Ingredient | Percentage (w/w) |
|---|---|
| Deionized water | 63.3% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Cinnamon oil | 0.4% |
| Citric acid | 1.0% |
| Orange oil | 0.2% |

TABLE 85
Soap Containing Cinnamon oil, Orange oil and Citric acid (CO-Cit 7)
(7 represent total oil 0.7%)

| Ingredient | Percentage (w/w) |
|---|---|
| Deionized water | 63.2% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Cinnamon oil | 0.5% |
| Citric acid | 1.0% |
| Orange oil | 0.2% |

TABLE 86
Soap Containing Orange oil and Citric acid (O-Cit 2)
(2 represents total oil 0.2%)

| Ingredient | Percentage (w/w) |
|---|---|
| Deionized water | 63.7% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Citric acid | 1.0% |
| Orange oil | 0.2% |

TABLE 87
Soap Containing Basil oil (“B oil”), Orange oil(“O oil”)
and Citric acid (BO-Cit 6)
(6 represents total oil 0.6%)

| Ingredient | Percentage (w/w) |
|---|---|
| Deionized water | 63.3% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Basil oil | 0.4% |
| Citric acid | 1.0% |
| Orange oil | 0.2% |

TABLE 88
Soap Containing Citronella oil (“CR oil”), Orange oil(“O oil”),
and Citric acid (CRO-Cit 6)
(6 represents total oil 0.6%)

| Ingredient | Percentage (w/w) |
|---|---|
| Deionized water | 63.3% |
| Methocel 40-101 | 0.1% |
| Pluronic F 87 Prill | 0.1% |
| Ucare Jr 30 | 0.1% |
| D,L Panthenol 50 W | 1.0% |
| Incromide oxide L | 3.0% |
| Crosultane C-50 | 3.0% |
| Montalene C 40 | 1.5% |
| 2-Phenoxy-ethanol | 1.0% |
| Glycerin | 2.0% |
| SDA-40B | 15.5% |
| Citronella oil | 0.4% |
| Citric acid | 1.0% |
| Orange oil | 0.2% |

### 15. EXAMPLE 11

Certain soaps prepared in Example 14 were tested for antimicrobial activity.

The following method was used. A mixture of 0.1 ml of 10⁷ cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. Soft soap® and Dial® soaps containing 0.15% triclosan was also tested similarly at the same time. The soap base without essential oils and Citric acid containing the culture were used as controls. The results, showing 30 second kill activity, are shown in Table 89.

TABLE 89

| Soap | Log10 reduction from control* |
|---|---|
| LG-Cit 4 | 3.9 |
| LG-Cit 6 | 4.2 |
| O-Cit 2 | 1.5 |
| LGO Cit 6 | 6.4 |

*Log 10 reduction from control bacterial counts (control counts ranges from 1 × 10⁶ to 5 × 10⁶)

These data show that when citric acid was used in combination with 0.4% LG oil+0.2% O oil (LGO-Cit 6) superior antibacterial activity was observed as compared to that of combination of Citric acid and LG oil 0.6% (LG-Cit 6) or the additive activity of Citric acid+0.4% LG oil (LG-Cit 4) and Citric acid+0.2% Orange oil (O-Cit 2).

### 16. EXAMPLE 12

Certain soaps described in Example 10 were tested for antimicrobial activity.

The following method was used. A mixture of 0.1 ml of 10⁷ cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. Soft soap® and Dial® soaps containing 0.15% triclosan was also tested similarly at the same time. The soap base without essential oils and citric acid containing the culture were used as controls. The results, showing 30 second kill activity, are shown in Table 90.

TABLE 90

| Soap | Log10 reduction from control* |
|---|---|
| LGO-Cit 6 | 6.4 |
| LGO-Cit 7 | 6.5 |
| CO-Cit 6 | 5.1 |
| CO-Cit 7 | 5.2 |
| BO-Cit 6 | 2.87 |
| CRO-Cit 6 | 4.57 |

*Log 10 reduction from control bacterial counts (ranges from 1 × 10⁶ to 3 × 10⁶.)

These data show that LGO-Cit Soaps were found to exhibit higher antibacterial activity compared to the other essential oil/citric acid combination soaps tested.

### 17. EXAMPLE 13

The following experiments were performed to evaluate the antibacterial activity of triclosan, LG oil, and combinations of triclosan and LG oil.

Patent application WO/2007/077573 by Mukhopadhyay et al describes an antimicrobial composition containing triclosan and an essential oil where the ratio of triclosan to the essential oil is 1:5 to 1:100 and the preferred ratio range is 1:10 to 1:90. In the example provided in United States Patent Application Publication No. 20050019431 by Modak et al., TC and essential oil at 1:1 ratio showed neither synergistic nor enhanced activity.

Triclosan (TC) is often used in personal care products at a concentration of 0.15-0.3%. In order to determine whether or not TC at this concentration would enhance the activity of essential oil at 0.4-0.7% which is the concentration used in various formulations described in this application, the antibacterial activity of soaps containing triclosan (“TC”); LG oil; or TC and LG oil at TC:LG weight ratios of 1:1.7 to 1:4.6 were evaluated.

To prepare the soaps, TC, LG oil or their combination were dissolved in SDA40 B alcohol and then added to Softsoap® (a formulation lacking triclosan), then diluted with water, where the amount of SDA40B alcohol used represented 5.5% of the final solution and the amount of Softsoap® used represented 92% of the final solution. Soft Soap® was used as the control in this study.

The following method was used. A mixture of 0.1 ml of 10⁸ cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The results are shown in Table 91.

TABLE 91

| Soap | Log10 reduction from control* |
|---|---|
| Softsoap ® + 0.15% TC | 0.70 |
| Softsoap ® + 0.3% TC | 0.81 |
| Softsoap ® + 0.5% LG oil | 0.76 |
| Softsoap ® + 0.7% LG oil | 0.75 |
| Softsoap ® + 0.15% TC + 0.5% LG oil | 0.74 |
| Softsoap ® + 0.15% TC + 0.7% LG oil | 0.92 |
| Softsoap ® + 0.3% TC + 0.5% LG oil | 0.77 |
| Softsoap ® + 0.3% TC + 0.7% LG oil | 0.77 |

*Log 10 reduction from control bacterial counts (ranges from 5.8 × 10⁷ to 6.4 × 10⁷ cfu)

These results indicate that no synergistic or enhanced effect was seen when triclosan was combined with LG oil at weight ratios falling within the range of 1:1.7 to 1:4.6.

### 18. EXAMPLE 14

The antibacterial activity of soaps containing 1) TC-LGO-Cit 6 at weight ratios within the range of between 1:3.3 and 1:4.7 (TC:LG) and between 1:1.4 and 1:2(LG:Citric acid) were evaluated against S. aureus. To prepare the soaps, triclosan/essential oil(s)/citric acid were dissolved in SDA40 B alcohol and added to Softsoap® (lacking triclosan) and diluted with water, so that the final concentration of alcohol was 5.5% and the final concentration of Softsoap® was 92 percent. A mixture of 0.1 ml of 10⁷ cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The results are shown in Table 92.

TABLE 92

| | Log10 |
|---|---|
| Soap | reduction from control* |
| Softsoap ® | — |
| Softsoap ® + 0.15% TC | 0.24 |
| Softsoap ® + 1% citric acid | 1.49 |
| Softsoap ® + 0.15% TC + ‘% citric acid | 2.01 |
| Softsoap ® + 0.15% TC + 0.5% LG oil + | 2.41 |
| 1% citric acid | |
| Softsoap ® + 0.15% TC + 0.4% LG oil + | 7.93 |
| 0.2% Orange oil + 1% citric acid | |

*Log 10 reduction from control bacterial counts (ranges from 1 × 10⁶ to 5 × 10⁶ cfu).

The foregoing data show that citric acid was found to enhance the activity of triclosan, and that addition of LG oil+O oil to a combination of triclosan and citric acid further enhanced the effect.

### 19. EXAMPLE 15

The following experiments were performed to compare the antibacterial activity of combinations of (i) lemongrass oil citric acid+triclosan; (ii) lemongrass oil+citric acid; and (iii) cinnamon oil-citric acid+triclosan, all in a Softsoap® base.

To prepare the soaps, triclosan/essential oil/citric acid were dissolved in SDA40 B alcohol and added to Softsoap® (lacking triclosan) and diluted with water, so that the final concentration of alcohol was 5.5% and the final concentration of Softsoap® was 92 percent. A mixture of 0.1 ml of 10⁸ cfulml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The results are shown in Table 93.

TABLE 93

| | Log10 |
|---|---|
| Soap | reduction from control* |
| Softsoap ® + 0.15% TC | 0.7 |
| Softsoap ® + 0.15% TC + 0.4% LG oil + | 7.93 |
| 0.2% Orange oil + 1% citric acid | |
| Softsoap ® + 0.4% LG oil + 0.2% Orange | 5.73 |
| oil + 1% citric acid | |
| Softsoap ® + 0.15% TC + 0.4% C oil + | 5.50 |
| 0.2% Orange oil + 1% citric acid | |
| Softsoap ® + 0.4% C oil + 0.2% Orange | 4.39 |
| Oil + 1% citric acid | |

*Log 10 reduction from control bacterial counts (ranges from 6.4 × 10⁷ to 9.9 × 10⁷ cfu)

The above data demonstrate, among other things, that LGO-Cit+Triclosan was found to be more effective than LGO-Cit and CO-Cit+Triclosan.

### 20. EXAMPLE 16

The following experiments were performed to evaluate the effect of adding various essential oil combinations, citric acid (0.5-0.7%), and SDA 40 B alcohol (5.5%) to commercial triclosan-containing soaps such as Dial® Soap and Softsoap® containing 0.15% Triclosan (“Dial® Soap-TC” and “Softsoap®-TC” respectively). A mixture of 0.1 ml of 10⁸ cfu/ml of S. aureus culture and 0.1 ml of bovine serum were placed in a sterile culture tube. 0.8 ml of the test soap formulation (or phosphate buffered saline as control) was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The formulations are shown in Tables 94-97. The results are shown in Table 98.

TABLE 94
Dial ® Soap TC-CO-Cit 7

| Ingredient | Percentage (w/w) |
|---|---|
| Cinnamon oil | 0.5 |
| Orange Oil | 0.2 |
| Citric acid | 1.0 |
| SDA 40 B alcohol | 5.5 |
| Dial ® Soap-TC | 92.8 |

TABLE 95
Dial ® Soap TC-LGO-Cit 7

| Ingredient | Percentage (w/w) |
|---|---|
| Lemon Grass oil | 0.5 |
| Orange Oil | 0.2 |
| Citric acid | 1.0 |
| SDA 40 B alcohol | 5.5 |
| Dial Soap ®-TC | 92.8 |

TABLE 96
Dial ® Soap TC-LG-Cit 5

| Ingredient | Percentage (w/w) |
|---|---|
| Lemon Grass oil | 0.5 |
| Citric acid | 1.0 |
| SDA 40 B alcohol | 5.5 |
| Dial ® Soap-TC | 92.8 |

TABLE 97
Softsoap ® TC-LGO-Cit 7

| Ingredient | Percentage (w/w) |
|---|---|
| Lemon Grass oil | 0.5 |
| Orange Oil | 0.2 |
| Citric acid | 1.0 |
| SDA 40 B alcohol | 5.5 |
| Softsoap ®-TC | 92.7 |

TABLE 98
Results

| | Log10 |
|---|---|
| Soap | reduction from control* |
| Dial ® Soap TC | 0.36 |
| Dial ® Soap-TC-CO-Cit 7 | 3.9 |
| Dial ® Soap-TC-LG-Cit 5 | 3.35 |
| Dial ® Soap-TC-LGO-Cit 7 | 5.09 |
| Softsoap ®-TC | 0.33 |
| Softsoap ®-TC-LGO-Cit 7 | 4.66 |
| Softsoap ®-TC + 1% citric acid | 2.64 |

*Log reduction from control bacterial counts (ranges from 2 0 × 10⁸ to 3.5 × 10⁸ cfu)

The above results indicate that citric acid was found to enhance the activity of soaps containing triclosan; the combination of citric acid and essential oils was found to increase the antimicrobial activity of soap containing triclosan, and superior antimicrobial action was associated with a combination of citric acid, lemongrass and orange oils, and triclosan.

### 21. EXAMPLE 17

The pH of soaps containing 1% citric acid typically ranges between 3.2-3.3. To determine whether or not the superior efficacy observed with the combination of essential oils and citric acid is due to the acidic pH, certain EO/citric acid containing-soaps were adjusted to pH 6.0 with 10 N sodium hydroxide and their antibacterial efficacy tested and compared to the corresponding soaps without pH adjustment. For the evaluation of antimicrobial activity, a mixture of 0.1 ml of 10⁷ cfulml of S. aureus culture (ATCC #6538) and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The results are shown in Table 99. (“Softsoap®-TC” is Softsoap® containing 0.15 percent triclosan).

TABLE 99

| Soap | Log10 reduction from control* |
|---|---|
| CO-Cit + Softsoap ®-TC pH 3.25 | 3.9 |
| CO-Cit + Softsoap ®-TC pH 6.0 | 3.25 |
| CLGO-Cit + Softsoap ®-TC pH 3.25 | 5.1 |
| CLGO-Cit + Softsoap ®-TC pH 6.0 | 5.65 |

*Log 10 reduction from control bacterial counts (ranges from 1 × 106 to 5 × 106.)

Conclusion: The efficacy was similar at both pH values tested. This indicates that the superior activity of essential oils and citric acid observed is not due to the acidic pH.

### 22. EXAMPLE 18

Household cleansers were prepared comprising citric acid (1-2%), alcohol, and either (i) lemongrass oil; (ii) a combination of lemongrass oil and pine oil; (iii) a combination of lemongrass oil and orange oil; or (iv)a combination of pine oil and orange oil. The antimicrobial effectiveness of these formulations were tested and compared to commercial Pinesol® cleanser (containing 8.7 percent pine oil and other ingredients including detergent and other cleaning agents) as a control.

TABLE 100
Stock solution of hard surface Disinfectant - LG-Cit 2

| Ingredient | Percentage (w/w) |
|---|---|
| Lemongrass oil | 2.0 |
| Citric Acid | 20.0 |
| SDA 40B alcohol | 76.5 |
| Pluronic Surfactant L-61 | 0.5 |
| Pluronic Surfactant F-127 | 0.5 |
| Pluronic Surfactant F-87 | 0.5 |

TABLE 101
Stock solution of hard surface Disinfectant. - LGP-Cit 4

| Ingredient | Percentage (w/w) |
|---|---|
| Lemongrass oil | 1.0 |
| Pine oil | 3.0 |
| Citric Acid | 20.0 |
| SDA 40B alcohol | 74.5 |
| Pluronic Surfactant L-61 | 0.5 |
| Pluronic Surfactant F-127 | 0.5 |
| Pluronic Surfactant F-87 | 0.5 |

TABLE 102
Stock solution of hard surface Disinfectant. - P-Cit 5

| Ingredient | Percentage (w/w) |
|---|---|
| Pine oil | 5.0 |
| Citric Acid | 20.0 |
| SDA 40B alcohol | 73.5 |
| Pluronic Surfactant L-61 | 0.5 |
| Pluronic Surfactant F-127 | 0.5 |
| Pluronic Surfactant F-87 | 0.5 |

After tenfold dilution of each stock solution the disinfectant contained the following percentages (w/w) of each ingredient.

TABLE 103

| Disinfectant | Ingredients | |
|---|---|---|
| Surface Disinfectant - LG cit 2 | 0.2% | Lemon grass oil |
| | 2% | Citric acid |
| | 7.65% | Alcohol |
| | 0.15% | surfactants |
| Surface Disinfectant - LG P cit 4 | 0.3% | Pine oil |
| | 0.1% | Lemon grass oil |
| | 2% | Citric Acid |
| | 7.45% | alcohol |
| | 0.15% | Surfactants |
| Surface Disinfectant - P cit 5 | 0.5% | Pine oil |
| | 2% | Citric acid |
| | 7.45% | alcohol |
| | 0.15% | surfactants |

To prepare the solution of Pinesol® to serve as control, as per the manufacturer's instruction, 6 ml of the Pinesol® containing 8.5% pine oil was diluted to 100 ml. This diluted sample contained 0.52% pine oil.

To test the antimicrobial activity, 0.1 ml of culture containing approximately 1×10⁷ colony forming units (“cfu”) of S. aureus per milliliter was spread evenly on the surface of 2.5×11 cm² tiles using a glass rod and left at room temperature for 10 minutes to dry. After 10 minutes 0.3 ml of the diluted surface disinfectant was spread evenly on the tiles with a sterile glass rod and left for another 10 minutes to dry. The tiles were rinsed with 9.6 ml of inactivating medium (BPBNS), which was collected for testing. The collected medium was serially diluted and 0.5 ml was plated onto TSA plates and incubated at 37° C. for 18-24 hours. The colonies on the plates were counted and the values converted to log₁₀.

TABLE 104

| | Log₁₀ reduction from control bacterial counts* | | | |
|---|---|---|---|---|
| | Disinfectant | Disinfectant | Disinfectant | |
| Organism | LG-Cit | LGP-Cit | P-Cit | Pine Sol |
| S. aureus | 3.56 | 1.89 | 0.81 | 2.4 |

*Log 10 reduction from control bacterial counts (ranges from 1 × 10⁶-5 × 10⁶)

These data indicate that a surface cleaner containing 0.2% LG oil and 2.0% Citric acid was found to be considerably more effective than a cleaner containing 0.5% Pine oil and 2% Citric acid as well as commercial Pinesol® Surface cleaner containing 0.52% Pine oil. The cleanser containing 0.3% Pine oil+0.1% LG oil+2% Citric acid was also found to be more effective than the one containing 0.5% Pine oil and 2% Citric acid.

### 23. EXAMPLE 19

The following stock solution was prepared.

TABLE 105
Stock Solution of hard surface Disinfectant. - POCit 7

| Ingredient | Percentage (w/w) |
|---|---|
| Pine oil | 5.0 |
| Orange oil | 2.0 |
| Citric Acid | 10.0 |
| SDA 40B alcohol | 53.5 |
| Pluronic Surfactant L-61 | 0.5 |
| Pluronic Surfactant F-127 | 0.5 |
| Pluronic Surfactant F-87 | 0.5 |

7.2% of the stock hard disinfectant was diluted with water to 100% before use. These diluted samples contained the following concentrations of active ingredients.

TABLE 106

| Disinfectant | Ingredients | |
|---|---|---|
| Surface Disinfectant - PO Cit 7 | 0.5% | Pine oil |
| | 0.2% | Orange oil |
| | 1% | Citric Acid |
| | 5.35% | alcohol |
| | 0.15% | Surfactants |

The following stock solution was prepared:

TABLE 107
Stock Solution of hard surface Disinfectant. - LGOCit 7

| Ingredient | Percentage (w/w) |
|---|---|
| Lemongrass oil | 5.0 |
| Orange oil | 2.0 |
| Citric Acid | 10.0 |
| SDA 40B alcohol | 53.5 |
| Pluronic Surfactant L-61 | 0.5 |
| Pluronic Surfactant F-127 | 0.5 |
| Pluronic Surfactant F-87 | 0.5 |

7.2% of the stock hard disinfectant was diluted with water to 100% before use. This diluted samples contained the following concentrations of active ingredients:

TABLE 108

| Disinfectant | Ingredients | |
|---|---|---|
| Surface Disinfectant - LGO Cit 7 | 0.5% | LG oil |
| | 0.2% | Orange oil |
| | 1% | Citric Acid |
| | 5.35% | alcohol |
| | 0.15% | Surfactants |

The method used in Example 18 was used to test antimicrobial activity.

TABLE 109

| | S. aureus | P. aeruginosa | E. coli |
|---|---|---|---|
| Log 10 reduction in | 0.6 | 5.1 | 5.1 |
| bacteria - PO-Cit 7 | | | |
| Log10 reduction in | 5.9 | 4.8 | 5.09 |
| bacteria - LGO-Cit 7 | | | |

*Log₁₀ reduction from control bacterial counts (ranges from 1 × 10⁶-5 × 10⁶)

The foregoing data indicate that LGO-Cit is effective against both gram positive and gram negative organisms while PO Cit is not very effective against the Gram positive organism S aureus.

### 24. EXAMPLE 20

The following experiments were carried out using either soap or surface disinfectants containing the EO(s)/citric acid combinations indicated. The test organism used was Candida albicans.

Where soap was employed, the following method was used. A mixture of 0.1 ml of 10⁷ cfu/ml of C. albicans culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The results, showing 30 second kill activity, are shown in Table 110.

Where surface disinfectant was employed, the following method was used. 0.1 ml of culture containing approximately 1×10⁷ colony forming units (“cfu”) of C. albicans per milliliter was spread evenly on the surface of 2.5×11 cm² tiles using a glass rod and left at room temperature for 10 minutes to dry. After 10 minutes 0.3 ml of the diluted surface disinfectant was spread evenly on the tiles with a sterile glass rod and left for another 10 minutes to dry. The tiles were rinsed with 9.6 ml of inactivating medium (BPBNS), which was collected for testing. The collected medium was serially diluted and 0.5 ml was plated onto TSA plates and incubated at 37° C. for 18-24 hours. The colonies on the plates were counted and the values converted to log₁₀.

TABLE 110

| Formulation | Log10 reduction from control* |
|---|---|
| CO-Cit 6 Soap | 1.02 |
| LGO-Cit 6 Soap | 1.27 |
| CO-Cit 7 Surface Disinfectant | 5.2 |
| LGO-Cit 7 Surface Disinfectant | 4.81 |

*Control counts range from 1 × 10⁶ to 5 × 10⁶

These results show that CO groups and LGO groups show similar activity against C. albicans.

### 25. EXAMPLE 21

Evaluation of the rapid antibacterial activity of various soap formulations was performed as follows.

Method of evaluation of rapidity of kill of soaps. The rapid antimicrobial efficacy of the soaps containing LG and various combinations were tested as follows. A mixture of 0.1 ml of 10⁹ cfulml of bacterial cultures and 0.1 ml of bovine serum was placed in a sterile culture tube. 0.8 ml of the test soap formulation was added to the tube and vortexed for 30 seconds. 9.0 ml drug neutralizing fluid (DNF) was added to the tube to neutralize the activity of the soap, this tube was vortexed and serially diluted with DNF. 0.5 ml of the diluted solution was plated on trypticase soy agar plates, incubated at 37° C. for 24-48 hrs and the colony counts were determined. The soap base without essential oils citric acid, secondary alcohol and Incroquat containing the culture were also tested. PBS was used as the control. LG-O-Cit 5 comprises 0.3 percent (weight/weight) lemongrass oil, 0.3 percent (weight/weight) orange oil, 1.0 percent (weight/weight) citric acid, 1.0 percent (weight/weight) 2-phenoxyethanol and 15 percent (weight/weight) SDA-40B alcohol. LG-O-Cit 4 comprises 0.3 percent (weight/weight) lemongrass oil, 0.1 percent (weight/weight) orange oil, 1.0 percent (weight/weight) citric acid, 1.0 percent (weight/weight) 2-phenoxyethanol and 15 percent (weight/weight) SDA-40B alcohol.

The amount of alkanediol, where present, is 0.3 percent (weight/weight). The complete formulations for the soaps specified are set forth in section 4.6, above. The results are shown in Table 111 below.

TABLE 111
Enhancement of the antibacterial activity of LG-O-Cit composition
by 0.3% of alkanediols (Test Organism: S. aureus)

| Soap formulations | Log10 reduction from control |
|---|---|
| Base | 1.8 |
| LG-O-Cit 5 | 3.7 |
| 1,2 decanediol (0.3%) | 0.6 |
| LG-O-Cit 5 + 1,2 decanediol | 4.5 |
| LG-O-Cit 4 | 3.6 |
| LG-O-Cit 4 + 1,2 decanediol | 4.8 |
| LG-O-Cit 4 + 1,2 dodecanediol | 4.5 |
| LG-O-Cit 4 + 1,2 Tetradecanediol | 4.5 |

*Log₁₀ reduction from Control bacterial counts (ranges from 2 × 10⁸-5 × 10⁸)

The results shown in Table 99 indicate that the alkanediols tested enhanced the antibacterial activity of LG+O oil and citric acid disinfectant composition at a concentration of 0.3 percent (weight/weight).

### 26. EXAMPLE 22

The method described in Section 25, above, was used to evaluate the antibacterial activity of soap formulations comprising 0.5 percent of alkanediols. LG-O-Cit 4A comprises 0.3 percent (weight/weight) lemongrass oil, 0.1 percent (weight/weight) orange oil, 1.0 percent (weight/weight) citric acid, 1.0 percent (weight/weight) 2-phenoxyethanol and 17 percent (weight/weight) SDA-40B alcohol. The amount of alkanediol, where present, is 0.5 percent (weight/weight). The complete formulations for the soaps specified are set forth in section 4.6, above. The results are shown in Table 112 below.

TABLE 112
Enhancement of the antibacterial activity of LG-O-Cit A
Composition by 0.5% of alkanediols
Rapid antimicrobial activity (30 second Kill)
(Test Organism S. aureus)

| Soap formulations | Log10 reduction from control |
|---|---|
| Base | 0.8 |
| LG-O-Cit 4A | 4.1 |
| 1,2 decanediol(0.5%) | 1.4 |
| LG-O-Cit 4A + 1,2 decanediol | 6.0 |
| LG-O-Cit 4A + 1,2 dodecanediol | 6.1 |
| LG-O-Cit 4A + 1,12 dodecanediol | 6.0 |
| LG-O-Cit 4A + 1,2 Tetradecanediol | 6.0 |
| LG-O-Cit4A + 0.25% 1,2decanediol + | 6.0 |
| 0.25%1,12Dodecanediol | |
| Cn-O-Cit4A | 3.7 |
| Cn-O-Cit4A + 1,2 decanediol | 4.9 |

(pH of all the soaps ranged from 4.5-4.6)
*Log₁₀ reduction from Control bacterial counts (ranges from 2 × 10⁸-5 × 10⁸)

The results shown in Table 112 indicate that alkanediols at 0.5% concentration showed significant enhancement of the antibacterial activity of LG+O oil+citric acid or Cn+O oil and citric acid disinfectant composition.

### 27. EXAMPLE 23

To evaluate the effect of decanediol on the antibacterial activity of citric acid or citric acid in combination with essential oils, the following experiments were performed. The compounds indicated below were incorporated into soft soap lacking triclosan and the activity was evaluated. Activity was measured as described in Section 25, EXAMPLE 22. The results are shown in Table 113.

TABLE 113
Rapid antimicrobial activity (30 second Kill)
(Test Organism S. aureus)

| | Log₁₀ reduction |
|---|---|
| Soap formulations (% w/w) | from control |
| Plain Soft soap | 0.2 |
| 0.5 decanediol | 1.4 |
| 1.0 citric acid | 1.3 |
| 0.5 decanediol + 1 citric acid | 6.5 |
| 0.3 + 0.1 LG + O | 0.1 |
| 0.5 decanediol + 1 citric acid + 0.3 + 0.1 LG + O | 7.0 |
| 0.25 decanediol + 0.5 citric | 4.7 |
| 0.15 + 0.06 LG + O | 0.1 |
| 0.25 decanediol + 0.5 citric + 0.15 + 0.06 LG + O | 5.6 |

*Log₁₀ reduction from PBS(Control) bacterial counts (ranges from 7 × 10⁷-1 × 10⁸)

The results shown in Table 113 indicate that decanediol and citric acid exhibit synergistic activity, and that further addition of essential oil enhances the activity. The use of decanediol+citric acid+essential oils in soap even at low concentrations was found to show superior antibacterial activity.

### 28. EXAMPLE 24

To determine the effect of LG-O-Cit-1,2 decanediol on the antibacterial activity of triclosan-containing soap, the following experiments were performed.

Dial® soap containing 0.15% Triclosan (Dial-T Soap) was used for this test. The following formulation was prepared. The antibacterial activity was then tested using the method set forth in Section 25, EXAMPLE 21. The results are shown in Table 114.

TABLE 114
Dial ®-T Soap Containing LG-O-Cit 4 and 0.5% 1,2 decanediol

| Ingredient | Percentage (w/w) |
|---|---|
| Dial ®-T soap | 90.0 |
| SDA 40B | 8.1 |
| Lemon grass oil | 0.3 |
| Orange oil | 0.1 |
| Citric acid | 1.0 |
| 1,2 decanediol | 0.5 |

Original pH was 3.2 pH adjusted to 4.5 with 10.N NaOH

TABLE 115
Enhancement of the activity of Triclosan by LG-O-Cit-1,2
Decanediol Rapid antimicrobial activity (30 second Kill)
(Test Organism S. aureus)

| | Log₁₀ reduction |
|---|---|
| Soap formulations | from control |
| Dial ®-T soap | 0.7 |
| Dial ®-T Soap + LG-O-Cit 4 | 5.5 |
| Dial ®-T Soap + LG-O-Cit4-0.5% | 8.0 |
| 1,2 decanediol | |

*Log₁₀ reduction from Control bacterial counts (ranges from 2 × 10⁸-5 × 10⁸)

The foregoing results indicate that decanediol enhances the activity of Dial®-T Soap+LG-O-Cit 4.

### 29. EXAMPLE 25

The antibacterial activity of LG-O-CitA-D-T Lotion, having the following formulation, was tested in a pigskin model.

TABLE 116

| Ingredient | Percentage (w/w) |
|---|---|
| Water | 65.6 |
| U Care-JR 30M | 0.25 |
| PolyoxWSR-205 | 0.1 |
| Incroquat TMS Behenyl | 2.0 |
| Isopropyl myristate | 1.0 |
| Acetulan | 1.0 |
| Vitamin E | 0.2 |
| Zinc stearate | 0.2 |
| Polawax NF | 2.75 |
| Glycerin | 2.0 |
| Allantoin | 0.2 |
| Dimethicone copolyol (Q2- | 2.5 |
| 5220) | |
| Citric acid | 1.0 |
| 1,2 decanediol | 0.5 |
| Tocopheryl acetate | 0.5 |
| Glyceryl stearate (Arlacel165) | 1.0 |
| Butylene glycol | 3.0 |
| SDA-40-B | 15 |
| Lemongrass oil | 0.5 |
| Tea tree oil | 0.5 |
| Orange oil | 0.1 |
| 1,2 Decanediol(Symclairol) | 0.5 |
| Triclosan | 0.3 |
| (pH adjusted to 4.5-5.0) | |

The pigskin model assay was as follows. Six sets of 3×3 cm² pig skin each mounted on a petriplate were rinsed in 70% isopropanol , and air dried. One piece of the pair was contaminated with 30 μl of 10⁸ cfu of MRSA culture; the two pieces were then rubbed against each other for 30 seconds , and left at 37° C. to dry for one hour. 3 pairs were used for control and another 3 pairs were used for the test, which was as follows.

To one piece of the pair from the control, 0.1 gm of placebo cream (same as LG-O-Cit4-D (above) without SDA-40-B, lemongrass oil, tea tree oil, orange oil, 1,2 decanediol (Symclairol) was applied, and rubbed against the other piece for 15 seconds and left at 37° C. for 1 hour. The same procedure was repeated with the test skins in which LG-O-CitA-D-T was applied. Following this, 0.2 ml dilution media (DM) was added to one skin piece and both pieces rubbed again for 15 seconds. The surviving organisms were recovered from the skin by rinsing each piece with 9.9 ml of DM. The washing fluid from both pieces was collected in one petri dish, mixed and transferred to a culture tube from which further serial dilutions were made. Aliquots from the dilutions were plated on TSA plates and incubated for 24-48 hours at 37° C. before colony counts (baseline counts) were determined. The results are shown in Table 117.

TABLE 117
Reduction of Bacterial growth I hour post treatment

| | | Log₁₀ reduction |
|---|---|---|
| Treatment | Bacterial counts | from |
| cream | (cfu/skin) | control counts |
| PBS | 2.2 × 10⁶ | — |
| Placebo cream (control) | 2.0 × 10⁶ | — |
| LG-O-Cit A-D-T Lotion | 7.6 × 10³ | 2.37 |

### 30. EXAMPLE 26

The antibacterial activity of preservative compositions was evaluated.

TABLE 118
Preservative composition A

| Ingredient | Percentage (w/w) |
|---|---|
| Lemongrass oil | 10 |
| Farnesol | 10 |
| Orange oil | 5 |
| lactic acid | 7 |
| 1,2 decanediol | 7 |
| SDA 40 B alcohol | 61 |

TABLE 119
Preservative composition B

| Ingredient | Percentage (w/w) |
|---|---|
| Lemongrass oil | 15 |
| Farnesol | 15 |
| Orange oil | 10 |
| lactic acid | 10 |
| SDA 40 B alcohol | 50 |

TABLE 120
Preservative composition C

| Ingredient | Percentage (w/w) |
|---|---|
| Farnesol | 17 |
| Citric acid | 7 |
| 1,2 decanediol | 7 |
| SDA 40 B alcohol | 69 |

TABLE 121
Preservative composition D

| Ingredient | Percentage (w/w) |
|---|---|
| Lemongrass oil | 15 |
| Orange oil | 5 |
| Lactic acid | 10 |
| 1,2 decanediol | 20 |
| 1,2 Octanediol | 20 |
| SDA 40 B alcohol | 30 |

TABLE 122
Preservative composition E

| Ingredient | Percentage (w/w) |
|---|---|
| Lemongrass oil | 15 |
| Orange oil | 5 |
| Lactic acid | 10 |
| 1,2 Octanediol | 40 |
| SDA 40 B alcohol | 30 |

The pH of these solutions are adjusted to 5.0. 0.5-5.0% of these preservatives can be used in various formulations.

Evaluation of the Preservative efficacy of Composition A and B. The following Cream base was prepared to incorporate the preservative before testing.

TABLE 123
Preservative composition F

| Ingredient | Percentage (w/w) |
|---|---|
| Water | 70.24 |
| Ucare JR 40 | 0.3 |
| Polowax | 3.0 |
| Incroquat Behenyl TMS | 3.0 |
| Petroleum jelly | 5.0 |
| Stearyl alcohol | 7.0 |
| Propylene glycol | 2.0 |
| Isopropyl myristate | 4.0 |
| Sorbitan oleate | 2.0 |
| Polyoxyl 40 stearate | 2.0 |

An overnight culture of bacteria grown in Trypticase Soy Broth (TSB) was diluted with TSB to obtain 10⁸ CFU organism/ml. For the test samples, 2% of the preservative was added to 10 grams of the cream and mixed well. From this sample, 1 gram aliquots were placed into 10 ml sterile plastic culture tubes and 0.1 ml (100 microliters) of the test inoculum was added and vortexed until uniformly blended. The tubes were then placed into incubators at 37° C. All tubes were incubated for a total of 3 days. At the end of the incubation period 9.0 ml of Butterfield Phosphate Buffered solution with neutralizer was added to the incubated cultured sample and vortexed until completely mixed. The samples were serially diluted and then plated in Trypticase soy agar (TSA). the plates were incubated at 37° C. temperature for 24-48 hours and the counts were read. The results are shown in Table 124, below.

TABLE 124
Log₁₀ Reduction from control growth

| | S. aureus | P. aeruginosa |
|---|---|---|
| Control | — | — |
| Preserv A | 7.8 | 8.0 |
| Preserv B | 6.7 | 4.0 |

Control growth for S. aureus and P aeruginosa are 6.5×10⁸ and 1×10⁸ cfu/gm respectively.

### 31. EXAMPLE 27

The following experiments were performed to evaluate wound dressings impregnated with essential oils, citric acid and decanediol.

TABLE 125
Antimicrobial Impregnation solution

| Ingredient | Percentage (w/w) |
|---|---|
| Lemongrass oil | 0.3 |
| Orange oil | 0.1 |
| Tea tree oil | 0.5 |
| Calandula oil | 0.5 |
| Citric acid | 1.0 |
| Olive oil | 5.0 |
| Propylene glycol | 10 |
| Decanediol | 0.5 |
| SDA 40 B alcohol | 51.7 |
| U care JR 30 | 0.4 |
| Water | 30 |

TABLE 126
Antimicrobial/anti inflammatory Impregnation solution

| Ingredient | Percentage (w/w) |
|---|---|
| Lemongrass oil | 0.3 |
| Orange oil | 0.1 |
| Tea tree oil | 0.5 |
| Calandula oil | 0.5 |
| Citric acid | 1.0 |
| Olive oil | 5.0 |
| Propylene glycol | 10 |
| Decanediol | 0.5 |
| SDA 40 B alcohol | 51.0 |
| U care JR 30 | 0.4 |
| Curcumin | 0.3 |
| Water | 29.7 |

Wound dressings (Dukal non adherent pad) were dipped into the antimicrobial impregnation solution and dried for 24 hours. The dressings were cut into 1 cm² and the zones of inhibition against various organisms were determined.

Zones of inhibition test. 1×1 cm² piece of each dressing was placed on Trypticase soy agar plate seeded on the surface with 0.3 mL of 10⁸ colony forming units (CFU)/mL) of the test organism. The plates were incubated at 37° C. for 24 hours. The zone of inhibition around the catheter segments, excluding the diameter of patch was measured. The results are shown in Table 127.

TABLE 127
Antimicrobial Impregnation solution

| Organism | Zone of inhibition (mm) |
|---|---|
| S. aureus | 7.0 |
| MRSA | 8.0 |
| P. aeruginosa | 5.0 |
| C. albicans | 9.0 |

### 32. EXAMPLE 28

The following experiment was performed to evaluate the efficacy of creams containing preservative compositions.

Creams containing 2.0%-3.0% of preservative compositions were prepared and tested according to the following method.

TABLE 128
Formulation

| Ingredients | Percentage (w/w) |
|---|---|
| Water | 70.24 |
| Ucare JR 40 | 0.3 |
| Polowax | 3.0 |
| Incroquat Behenyl TMS | 3.0 |
| Petroleum Jelly | 5.0 |
| Stearyl alcohol | 7.0 |
| Propylene glycol | 2.0 |
| Isopropyl myristate | 4.0 |
| Sorbitan oleate | 2.0 |
| Polyoxyl 40 stearate | 2.0 |

An overnight culture of bacteria grown in Trypticase Soy Broth (TSB) was diluted with TSB to obtain 108 CFU organism/ml. For the test samples, 2% of the preservative was added to 10 grams of the cream and mixed well. From this sample, 1 gram aliquots were placed into 10 ml sterile plastic culture tubes and 0.1 ml (100 microliters) of the test inoculum was added and vortexed until uniformly blended. The tubes were then placed into incubators under the following temperatures: 300° C. for Aspergillus niger and 370° C. for the remaining three microbes. All tubes were incubated for a total of 3 days. At the end of the incubation period, 9.0 ml of Butterfield Phosphate Buffered solution with neutralizer was added to the incubated cultured sample and vortexed until completely mixed. The samples were serially diluted and then plated in Trypticase soy agar (TSA). The plates were incubated at 370° C. temperature for 24-48 hours, and the counts were read. Placebo cream was tested similarly and used as the control. The following Table reflects the results of the testing.

TABLE 129
Log10 Reduction from control growth

| Preservative | S. aureus | P. aeruginosa | Aspergillus niger |
|---|---|---|---|
| F | 6.8 | 4.65 | 5.0 |
| G | 7.3 | 5.8 | 5.1 |
| H | 5.5 | 5.0 | 3.0 |
| I | 6.5 | 5.0 | 4.9 |
| J | 5.5 | 4.5 | 3.0 |
| K | 5.8 | 4.8 | 4.0 |
| L | 6.0 | 5.2 | 3.8 |
| M | 6.8 | 5.5 | 5.0 |

Control growth for S. aureus and P aeruginosa were 6.5×10⁸ and 1×10⁸ cfu/gm, respectively, and for A. niger was 6×10⁴-1×10⁵. Based on these results, all of the above preservative compositions were effective.

Various patent and non-patent publications are cited herein, the contents of which are hereby incorporated by reference in their entireties.

We claim:

1. A personal care product composition comprising (i) one or more essential oil or individual constituent thereof at a concentration between about 0.1 and 1.2 percent (weight/weight); and (ii) a fruit acid at a concentration between about 0.5 and 1.5 percent (weight/weight); having one or more characteristic selected from the group consisting of:
(a) the composition further comprises one or more than one component selected from the group consisting of emollients, thickening agents, gelling agents, humectants, silicone polymers, and hydrogels;
(b) the composition is suitable for use as a personal care product selected from the group consisting of a bar soap, a liquid hand soap, a hand sanitizer, wound care product, a body wash, an acne treatment, a shampoo, a hair conditioner, a cosmetic, a deodorant, a body lotion, a hand cream, a topical cream, an aftershave lotion, a skin toner, a mouth wash, a toothpaste, a sunscreen lotion, a baby cleansing wipe, and a diaper cream; and
(c) regular exposure of the skin to the composition does not produce skin irritation in a normal subject.

2. The personal care product composition of claim 1, wherein (i) the one or more essential oil or individual constituent thereof is present at a concentration between about 0.1 and 1 percent (weight/weight); (ii) the fruit acid is present at a concentration between about 0.125 and 1 percent (weight/weight); and further comprising alcohol at a concentration between about 5 and 20 percent (weight/weight).

3. The personal care product composition of claim 2, further comprising triclosan at a concentration between about 0.05 and 1 percent (weight/weight).

4. The personal care product composition of claim 1, 2 or 3, wherein the essential oil and/or constituent thereof is selected from the group consisting of lemongrass oil, an individual constituent of lemongrass oil, orange oil, an individual constituent of orange oil, cinnamon leaf oil, and individual constituent of cinnamon leaf oil, basil oil, and an individual constituent of basil oil.

5. The personal care product composition of claim 1, 2 or 3, wherein the fruit acid is citric acid.

6. The personal care product composition of claim 4, wherein the fruit acid is citric acid.

7. The personal care product composition of claim 1, further comprising an anti-inflammatory agent.

8. The personal care production composition of claim 7, wherein the anti-inflammatory agent is calendula oil at a concentration between about 0.3 and 0.7 percent (weight/weight).

9. A personal care product composition comprising:
(i) lemongrass oil or an individual constituent thereof;
(ii) orange oil or an individual constituent thereof;
(iii) fruit acid at a concentration between about 0.25 and 1 percent (weight/weight); and
(iv) alcohol at a concentration between about 5 and 20 percent (weight/weight),
wherein the total concentration of lemon grass oil or an individual constituent thereof and orange oil or an individual constituent thereof is between about 0.2 and 0.7 percent (weight/weight).

10. The personal care product composition of claim 9, further comprising triclosan at a concentration between about 0.05 and 1 percent.

11. The personal care product composition of claim 9 or 10, wherein the fruit acid is citric acid.

12. The personal care product composition of claim 9 or 10, which is suitable for use as a personal care product selected from the group consisting of a bar soap, a liquid hand soap, a hand sanitizer, a body wash, an acne treatment, a shampoo, a hair conditioner, a cosmetic, a deodorant, a body lotion, a hand cream, a topical cream, an aftershave lotion, a skin toner, a mouth wash, a toothpaste, a sunscreen lotion, a baby cleansing wipe, and a diaper cream.

13. The personal care product composition of claim 11, which is suitable for use as a personal care product selected from the group consisting of a bar soap, a liquid hand soap, a hand sanitizer, a body wash, an acne treatment, a shampoo, a hair conditioner, a cosmetic, a deodorant, a body lotion, a hand cream, a topical cream, an aftershave lotion, a skin toner, a mouth wash, a toothpaste, a sunscreen lotion, a baby cleansing wipe, and a diaper cream.

14. A surface cleaner composition comprising more than one essential oil or constituent thereof at a total concentration between about 0.1 and 1 percent (weight/weight), a fruit acid at a concentration of between about 1 and 2 percent (weight/weight), and an alcohol, at a concentration of between about 5 and 20 percent (weight/weight), where the essential oils or constituents thereof are selected from the group consisting of lemongrass oil, an individual constituent of lemongrass oil, orange oil, and individual constituent of orange oil, basil oil, an individual constituent of basil oil, pine oil, and an individual constituent of pine oil.

15. The surface cleaner composition of claim 14, further comprising a surfactant.

16. The surface cleaner composition of claim 14, comprising pine oil and orange oil.

17. The surface cleaner composition of claim 14, comprising pine oil and lemongrass oil.

18. The surface cleaner composition of claim 14, comprising lemongrass oil and orange oil.

19. A veterinary product composition comprising (i) one or more essential oil or individual constituent thereof at a concentration between about 0.1 and 1.2 percent (weight/weight); and (ii) a fruit acid at a concentration between about 0.5 and 1.5 percent (weight/weight); wherein the veterinary product composition is suitable for use as a product selected from the group consisting of a pet shampoo, a pet cleansing wipe, an ear cleaning liquid, a cage cleaner, a topical cream, a teat dip, or a pet body splash.

20. An antimicrobial composition comprising (i) between about 0.2 and 0.5 percent (weight/weight) of one or more essential oil selected from the group consisting of lemongrass, cinnamon oil, citronella oil, basil oil, orange oil and combinations thereof; (ii) a non-alkanediol alcohol solvent at a concentration between about 0.5 and 20 percent (weight/weight); (iii) an amount of alkanediol which increases the antimicrobial effect, and (iv) one or more fruit acid at a total concentration between about 0.125 and 2.0 percent (weight/weight).

21. The composition of claim 20, where the fruit acid is selected from the group consisting of citric acid and lactic acid.

22. The composition of claim 20, where the alkanediol is present at a concentration between about 0.3 and 1.0 percent (weight/weight).

23. The composition of claim 20, where the alkanediol is selected from the group consisting of dodecanediol, decanediol, nonanediol, octanediol, heptanediol, hexanediol and pentanediol.

24. The composition of claim 20 where the alkanediol has a carbon backbone of between 9 and 25 carbon atoms.

25. The composition of claim 24, where the alkanediol is selected from the group consisting of 1,9 Nonanediol, 1,2-Decanediol, 1,10-Decanediol, 1,11-Undecanediol, 1,2-Dodecanediol, 1,12 Dodecanediol, Cyclododecanediol, 1,13-Tridecanediol, 1,2-Tetradecanediol, 1,14-Tetradecanediol, 1,15-Pentadecanediol, 1,16-Hexadecanediol, 1,17-Heptadecanediol, 1,18-Octadecanediol, 1,19-Nonadecanediol, 1,20-Eicosanediol, 1,21-Heneicosanediol, 1,22-Docosanediol, 1,23-Tricosanediol, 1,24-Tetracosanediol, 1,25-Pentacosanediol.

26. The composition of claim 24, where the alkanediol is selected from the group consisting of 1,2-Decanediol, 1,10-Decanediol, 1,2-Dodecanediol, 1,12-Dodecanediol, Cyclododecanediol, 1,13-Tridecanediol, 1,2-Tetradecanediol, 1,14-Tetradecanediol and the most preferred alkanediols are 1,2-Decanediol, 1,2-Dodecanediol and 1,2-Tetradecanediol.

27. The composition of claim 20, where the non-alkanediol alcohol is an aliphatic alcohol.

28. The composition of claim 20, where the non-alkanediol alcohol is an aromatic alcohol.

29. The composition of claim 20, where the non-alkanediol alcohol is selected from the group consisting of methanol, ethanol, n-propanol, isopropyl alcohol, 2-methyl-2 propanol, hexanol, phenoxyethanol, benzyl alcohol, 1-phenoxy-2propanol, and phenethyl alcohol.

30. A personal care product selected from the group consisting of a bar soap, a liquid hand soap, a hand sanitizer, a body wash, an acne treatment, a shampoo, a hair conditioner, a cosmetic, a deodorant, a body lotion, a hand cream, a topical cream, an aftershave lotion, a skin toner, a mouth wash, a toothpaste, a sunscreen lotion, a baby cleansing wipe, and a diaper cream, comprising (i) between about 0.2 and 0.5 percent (weight/weight) of one or more essential oil selected from the group consisting of lemongrass, cinnamon oil, citronella oil, basil oil, orange oil and combinations thereof; (ii) a non-alkanediol alcohol solvent at a concentration between about 0.5 and 20 percent (weight/weight); (iii) an amount of alkanediol which increases the antimicrobial effect, and (iv) one or more fruit acid at a total concentration between about 0.125 and 2.0 percent (weight/weight).

31. A household cleaner comprising (i) between about 0.2 and 0.5 percent (weight/weight) of one or more essential oil selected from the group consisting of lemongrass, cinnamon oil, citronella oil, basil oil, orange oil and combinations thereof; (ii) a non-alkanediol alcohol solvent at a concentration between about 0.5 and 20 percent (weight/weight); (iii) an amount of alkanediol which increases the antimicrobial effect, and (iv) one or more fruit acid at a total concentration between about 0.125 and 2.0 percent (weight/weight).

32. A veterinary product comprising (i) between about 0.2 and 0.5 percent (weight/weight) of one or more essential oil selected from the group consisting of lemongrass, cinnamon oil, citronella oil, basil oil, orange oil and combinations thereof; (ii) a non-alkanediol alcohol solvent at a concentration between about 0.5 and 20 percent (weight/weight); (iii) an amount of alkanediol which increases the antimicrobial effect; and (iv) one or more fruit acid at a total concentration between about 0.125 and 2.0 percent (weight/weight).

33. A preservative composition comprising (i) between about 10 and 30 percent (weight/weight) of one or more essential oil, (ii) between about 3 and 15 percent (weight/weight), (iii) between about 0.5 and 70 percent (weight/weight) a non-alkanediol alcohol solvent, and (iv) between about 0 and 35 percent (weight/weight) of an alkanediol.

34. The preservative composition of claim 33, wherein between about 0.5 and 5.0 percent of the preservative composition is used in personal care products.

35. A veterinary product comprising (i) between about 0.1 and 5.0 percent (weight/weight) of one or more anti-irritant; (ii) between about 0.2 and 1.0 percent (weight/weight) of a vehicle containing gelling agent; (iii) between about 5 and 15 percent (weight/weight) of glycerin; (iv) between about 50 and 80 percent (weight/weight) water; (v) between about 0.2 and 2 percent antimicrobial agents comprising botanicals; (vi) between about 0.5 and 15 percent (weight/weight) of one or more alcohols; and (vii) between about 0.3 and 1 percent (weight/weight) of an alkanediol.
